(19) United States
(12) Patent Application Publication
(10) Pub. No.: US 2016/0128865 A1
(43) Pub. Date: May 12, 2016

(54) SYSTEM FOR PROVIDING TREATMENT TO A MAMMAL AND METHOD

(71) Applicant: Mark H. Lowe, Danville, CA (US)

(72) Inventor: Mark H. Lowe, Danville, CA (US)

(21) Appl. No.: 15/000,887

(22) Filed: Jan. 19, 2016

Related U.S. Application Data
(63) Continuation of application No. 13/441,761, filed on Apr. 6, 2012.
(60) Provisional application No. 61/472,596, filed on Apr. 6, 2011.
(60) Provisional application No. 61/472,598, filed on Apr. 6, 2011.
(60) Provisional application No. 61/472,602, filed on Apr. 6, 2011.

(51) Int. Cl. A61F 7/02; A61H 9/00

(52) U.S. Cl. CPC A61F 7/02; A61H 9/0092; A61F 2007/0056

(57) ABSTRACT

Therapy system for treatment of an animate body including core body cooling. The system includes a therapy wrap and a first therapy device for exchanging heat with the body. The system may include a second therapy device for delivering another therapy. The first therapy device may include a heat transfer device, a cooling or heating source, and a control unit. The heat transfer device may include a fluid bladder and a compressive bladder. The second therapy device may be an electric stimulation device. The therapy wrap is adapted to coordinate delivery of the thermal therapy and another therapy to the animate body. The therapy wrap may also be a sleeve for mounting components of the first and second therapy devices. Also disclosed are a method of administering a temperature-controlled treatment to an anatomical body part and method of making the therapy system.

### CROSS REFERENCE TO RELATED APPLICATIONS

This application is a continuation of U.S. patent application Ser. No. 13/441,761, filed Apr. 6, 2012, now U.S. Patent Application Publication No. 2013/0013033, which claims priority to U.S. Provisional Patent Application No. 61/472,596, filed on Apr. 6, 2011; U.S. Provisional Patent Application No. 61/472,598, filed on Apr. 6, 2011; and U.S. Provisional Patent Application No. 61/472,602, filed on Apr. 6, 2011, which are herein incorporated by reference in their entireties.

### FIELD OF THE INVENTION

The present invention relates generally to therapy of an animate body, and more particularly a system for providing temperature-controlled therapy to a mammal.

### INCORPORATION BY REFERENCE

All publications and patent applications mentioned in this specification are herein incorporated by reference for all purposes to the same extent as if each individual publication or patent application was specifically and individually indicated to be incorporated by reference.

### BACKGROUND OF THE INVENTION

Sudden cardiac arrest is a disruption of the heart's functioning that causes a lack of blood flow to vital organs. In a majority of instances, sudden cardiac arrest is manifested as an abnormal or chaotic heart rhythm, called arrhythmia. These instances are generally identifiable by the victim's immediate loss of pulse, loss of consciousness, and/or a cessation of breathing.

Sudden cardiac arrest has been attributed to over 350,000 deaths each year in the United States alone, making it one of the world's leading medical emergencies. Sudden cardiac arrest typically leads to death within a matter of minutes without medical intervention. Survival rates for sudden cardiac arrest in hospitals are around 40% and outside hospitals around 5%. The survival rate is even lower in locations where transportation to a hospital can be slow.

Thus, the treatment provided by the emergency medical response (EMS) team plays an important role in affecting patient outcomes. In addition to survivability, EMS care affects recovery and long term outcomes. For example, the first thirty minutes after cardiac arrest can be as critical as the hospital care over the succeeding hours and weeks. During this “golden half hour,” EMS technicians work to stabilize the patient en route to a hospital and minimize trauma to the brain and other body regions starved of blood flow.

Because most patients suffer cardiac arrest and other emergency medical events while away from the hospital, there is a need for treatment devices and methods effective outside the hospital. An example of such a device is the portable defibrillator. Portable defibrillators have long been a critical tool for reviving and stabilizing patients who have suffered cardiac arrest.

More recently it has been found that cooling the body and the brain immediately after various serious medical events, such as cardiac arrest, can significantly reduce the risk of loss of faculty and other long-term effects. It has also been found that cooling the body in other circumstances can improve patient outcomes, such as post-operative care, treatment of trauma, and emergency care for events beyond cardiac arrest. When starved of oxygen, heart cells begin to die in 20 minutes and brain cells begin to die almost immediately. However, it has been found that deep core cooling of the body can significantly delay the loss of cells, in some cases by hours.

Temperature-controlled therapy has long been practiced in the field in various settings include pre-hospital (e.g. EMS), clinical, physical therapy, and hospital settings. Thermal therapy commonly includes cooling and/or heating and applying compression to a traumatized area of a human body to facilitate healing and prevent unwanted consequences of the trauma. This form of therapy is commonly referred to as RICE (Rest, Ice, Compression and Elevation). RICE is also commonly used in sports medicine to reduce the risk of long-term damage to muscles and joints and/or alleviate pain and soreness.

Conventional temperature-controlled therapy involves applying ice bags or the like to a treatment area to provide deep cooling. Elastic wraps are often applied over the bags to keep them in place and provide compression to the body part. Ice bags and elastic wraps lack control and usually require a user to put the bag on and off to adjust cooling.

More sophisticated animate body heat exchangers have been developed recently. Temperature-controlled therapy systems commonly include a heat exchanger, a control unit for the heat exchanger, and a sleeve for positioning the heat exchanger on a body part to be treated. The control unit regulates delivery of a heat exchange fluid to the heat exchanger for circulation through a fluid bladder. Many systems also include a compressive mechanism such as a compliant gas pressure bladder that overlays the fluid bladder. The gas pressure bladder directs a compressive force to the fluid bladder to press the bladder against the body part to be subjected to heat exchange, as well as apply compression to the body part to reduce edema.

While existing temperature-controlled therapy systems have broad application for treatment of body parts, existing systems have limited effectiveness for core cooling or heating of an entire warm blooded body. Thermoregulation is the ability of an organism to keep its body temperature within certain boundaries when exposed to different temperature environments. The process of maintaining this dynamic state of stability between an animal's internal environment and its external environment is called homeostasis. When external cooling is applied to the body, it self-regulates to maintain its core temperature. Too much cooling can cause hypothermia, cold burns, and edema. Insufficient cooling will not cause the desired decrease in core temperature. The same applies to heating therapy. In other words, the temperature-controlled therapy must overcome the body's natural defenses without causing injury. Internal cooling methods, such as introduction of a cooling fluid into the bloodstream, more directly cool the body but suffer from the same problems. Additionally, internal cooling is invasive and thus suffers from the increased risk of complications, patient discomfort, and a likely increase in recovery time.

Additionally, existing therapy systems lack features to coordinate treatment with other devices. For example, existing thermal therapy devices typically completely cover a body part and do not allow access to the body for administration of other treatments. Similarly, existing external portable defibrillator paddles inhibit the wrapping of the body with a thermal therapy device.

Accordingly, there is a need for improved systems and methods for administering treatment to body.

There is a need for improved systems and methods for heating, cooling, and/or compressing a body in need of treatment. There is a need for systems and methods for core body cooling and heating.

There is the need to provide a temperature-controlled therapy system with improved effectiveness. There is a need for a temperature-controlled therapy system with improved patient comfort and/or reduced risks of injury to the body part treated. There is the need for an easy-to-use temperature-controlled therapy system applicable in a variety of settings and environments.

There remains a need to provide improved temperature-controlled therapy apparatus and methods for their use. These and other problems are overcome by the invention disclosed herein.

### SUMMARY OF THE INVENTION

The present invention involves improvements in heat transfer therapy apparatus and avoids disadvantages in the prior art.

Various aspects of the invention are directed to a system for providing treatment to an animate body requiring treatment, the system comprising a first therapy device adapted to provide temperature-controlled treatment to an animate body, the first therapy device including a heat transfer device adapted to transfer heat with the animate body; a second therapy device adapted to provide another treatment to the animate body and including a working element; and a therapy wrap for attaching the heat transfer device and working element to at least a portion of the animate body. In some embodiments, the first therapy device and the second therapy device are contained within an integrated system. In some embodiments, the system further includes a controller configured to execute a temperature-controlled treatment protocol with the first therapy device, wherein the temperature-controlled treatment protocol includes a cooling phase for lowering the temperature of the animate body, a maintenance phase for maintaining the temperature of the animate body, and a warming phase for increasing the temperature of the animate body.

Various aspects of the invention are directed to a system for providing therapy to an animate body, the system comprising a first therapy device adapted to provide temperature-controlled treatment to an animate body, the first therapy device including a heat transfer device adapted to transfer heat with the animate body and a control unit. In various embodiments, the system is configured to reduce the core body temperature of the animate body. In various embodiments, the system comprises a plurality of heat transfer devices. In various embodiments, each of the plurality of heat transfer devices is configured for different heat exchange rates with the body. In some embodiments, the plurality of heat transfer devices includes a first heat transfer device adapted to cover the torso of the animate body and a second heat transfer device adapted to cover an extremity of the animate body, wherein the first heat transfer device is configured to cool the torso of the animate body and modulate the core temperature of the animate body and the second heat transfer device is configured to warm the extremity.

Various aspects of the invention are directed to a method for treating an animate body in need of treatment, the method comprising applying a therapy wrap to a portion of an animate body; connecting the therapy wrap to a first therapy device comprising a heat transfer device adapted to transfer heat with the animate body; connecting the therapy wrap to a second therapy device including a working element and adapted to provide treatment to the animate body; cooling the body using the first therapy device; and stopping the cooling when an endpoint is detected. In various embodiments, the stopping comprises gradually restoring the therapy wrap to a normal temperature. Various embodiments of the method further include modulating the core temperature of the animate body.

The wrap and method of the present invention have other features and advantages which will be apparent from or are set forth in more detail in the accompanying drawings, which are incorporated in and form a part of this specification, and the following Detailed Description of the Invention, which together serve to explain the principles of the present invention.

### BRIEF DESCRIPTION OF THE DRAWINGS

FIG. 1 is a simplified schematic view of a therapy system in accordance with the invention, comprising a thermal therapy device and supplemental therapy device.

FIG. 2 is a block diagram of a portion of the temperature-controlled therapy device of FIG. 1, illustrating a control unit comprising a reservoir and ice box.

FIG. 3 is a block diagram of a control unit similar to that of FIG. 2, illustrating a control unit comprising a reservoir and refrigeration unit.

FIG. 4 is a block diagram of the circuit design of the thermal therapy device of FIG. 1.

FIG. 5 is a block diagram of an electric stimulation device for use with the system of FIG. 1.

FIG. 6 is a block diagram of the electric stimulation device of FIG. 5, comprising a portable defibrillator.

FIG. 7 is a schematic view of the electric stimulation device of FIG. 5.

FIG. 8 is an isometric illustration of the therapy wrap of FIG. 1 on a human.

FIG. 9 is an isometric illustration of the therapy wrap of FIG. 8, illustrating coupling of the wrap to other components of the electric stimulation device and temperature-controlled therapy device.

FIG. 10 is a top plan view of the therapy wrap of FIG. 8.

FIGS. 11A to 11E are front views of several exemplary therapy wraps similar to that of FIG. 8. FIG. 11A is a front view of a therapy wrap comprising a plurality of cooling regions. FIG. 11B is a front view of a therapy wrap comprising a thermal therapy device including a heat transfer device overlaying an electrode assembly. FIG. 11C is a front view of a therapy wrap comprising a thermal therapy device including a heat transfer device with a cut-out for an electrode assembly. FIG. 11D is a front view of a therapy wrap comprising a thermal therapy device including a plurality of heat transfer devices and an electrode assembly. FIG. 11E is a front view of a therapy wrap comprising a thermal therapy device including a plurality of heat transfer devices and an electrode assembly.

FIGS. 12A to 12E are schematic cross-sectional views of several therapy wraps similar to that of FIG. 8. FIG. 12A is a cross-sectional view of a therapy wrap comprising a fluid bladder, a gas pressure bladder, and a supplemental therapy device, illustrating the supplemental therapy device in a wall of the therapy wrap sleeve. FIG. 12B is a cross-sectional view of a therapy wrap comprising a fluid bladder, a gas pressure bladder, and a supplemental therapy device, illustrating the supplemental therapy device positioned inside the sleeve adjacent the gas pressure bladder and fluid bladder. FIG. 12C is a cross-sectional view of a therapy wrap comprising a fluid bladder, a gas pressure bladder, and a supplemental therapy device, illustrating the supplemental therapy device adjacent the therapy wrap sleeve. FIG. 12D is a cross-sectional view of a therapy wrap comprising a fluid bladder, a gas pressure bladder, and a supplemental therapy device, illustrating the supplemental therapy device positioned in a pocket along a wall of the therapy wrap sleeve. FIG. 12E is a cross-sectional view of a therapy wrap comprising a fluid bladder, a gas pressure bladder, and a supplemental therapy device, the supplemental therapy device being adapted for low level electrical stimulation.

FIG. 13 is a plan view of a fluid bladder for use with the therapy wrap of FIG. 8, illustrating fluidic channels in the fluid bladder and jumper connections along the channel.

FIG. 14 is a flowchart of a method of operating the therapy system of FIG. 1 in accordance with the invention.

FIGS. 15A to 15C are illustrations of several cooling sources for use with the temperature-controlled therapy device of FIG. 1. FIG. 15A is an illustration of a cooling source comprising a thermoelectric cooler. FIG. 15B is an illustration of a cooling source comprising a chemical cooling device. FIG. 15C is an illustration of a cooling source comprising an artificial ice pack.

FIG. 16 is an isometric illustration of a therapy wrap similar to that of FIG. 8 on a human during exercise, the therapy wrap comprising a vest and a skull cap.

FIG. 17 is an enlarged rear view of the cap of FIG. 16.

FIG. 18 is a front illustration of a therapy wrap similar to that of FIG. 8, the wrap adapted for positioning around a torso or mid-section of a human body.

FIG. 19 is a front illustration of a therapy wrap similar to that of FIG. 8, on a human leg and mid-section.

FIG. 20 is a side view of the wrap of FIG. 19.

### DETAILED DESCRIPTION OF THE INVENTION

Before the present invention is described, it is to be understood that this invention is not intended to be limited to particular embodiments or examples described. Further, when referring to the drawings, like numerals indicate like elements.

Unless expressly stated otherwise, the terms used herein are to be understood as used by one of ordinary skill in the art. In various respects, use of the singular in connection with the terms herein includes the plural and vice versa.

“Body” is to be understood as used in the medical and biological fields and generally includes any animate body including, but not limited to, mammals. In various respects, “body” refers to human or equine patients. In various respects “body part” and “body” are used interchangeably to refer to an animate subject in need of treatment. In various respects, “body part” refers to a part of a body in direct communication with a therapy system as described herein.

“Core cooling” is to be understood as generally used in the medical and biological fields, and in various respects, refers to application of cooling therapy to decrease the body temperature. In various respects, “core cooling” refers to cooling of the internal body temperature of a human below 95 degrees F. In various respects, “core cooling” refers to cooling of the internal body temperature of a human to between about 90 degrees F. and about 94 degrees F.

“Body temperature” and “internal body temperature” refer to the internal temperature or core temperature of a respective body as understood in the medical art.

As used herein, the “average temperature” of the wrap refers to the average of the wrap inlet temperature and the wrap outlet temperature.

“Temperature delta” refers to the difference between the wrap outlet temperature and the wrap inlet temperature. One will appreciate that in most cases the temperature delta through the wrap depends on the fluid flow rate, the heat load, and the specific heat of the thermal fluid.

“Maximum temperature” and “minimum temperature” generally refer to the maximum and minimum temperatures in a respective element, and in various respects, within the fluid bladder of the wrap.

“Heat transfer fluid” is to be understood as generally used in the art, and in various respects, refers to the fluid circulated in the heat transfer device for exchanging heat with the subject animate body. “Heat transfer fluid”, “heat transfer medium”, “heat exchange medium” and “heat exchange fluid” are used somewhat interchangeably. In various respects, “heat exchange medium” refers to a medium or cooling source through which the heat transfer fluid is passed to lower its temperature before circulating in the heat transfer device.

As used herein, “supplemental” generally means additional or auxiliary. “Supplemental therapy device” generally refers to a device that is additional to the thermal therapy device. In various respects, “supplemental therapy device” refers to a device that provides a therapy to the body that is different from thermal therapy. In various respects, the supplemental therapy device is an extension of the thermal therapy device. In various respects, the thermal therapy device is an extension of the supplemental therapy device. The supplemental therapy device and thermal therapy device may be distinct or they may overlap, such as by sharing components. In various respects, “supplemental therapy device” and “second therapy device” are used interchangeably.

As used herein, “working element” refers to an element or component of the larger device that is configured to act on the body in connection with a respective treatment. In various respects, “working element” refers to an element that directly interacts with the body. For example, the working element of a cooling therapy device is the cooling source that exchanges heat with the body. Similarly, the working element of a defibrillator is the electrode structure applied to the body.

As used herein, “isolated” is to be understood as generally used in the mechanical and electrical arts. In various respects, “isolated” refers to setting apart a subject element from another to essentially eliminate influence on the function of the subject element.

### I) General Description of Therapy System

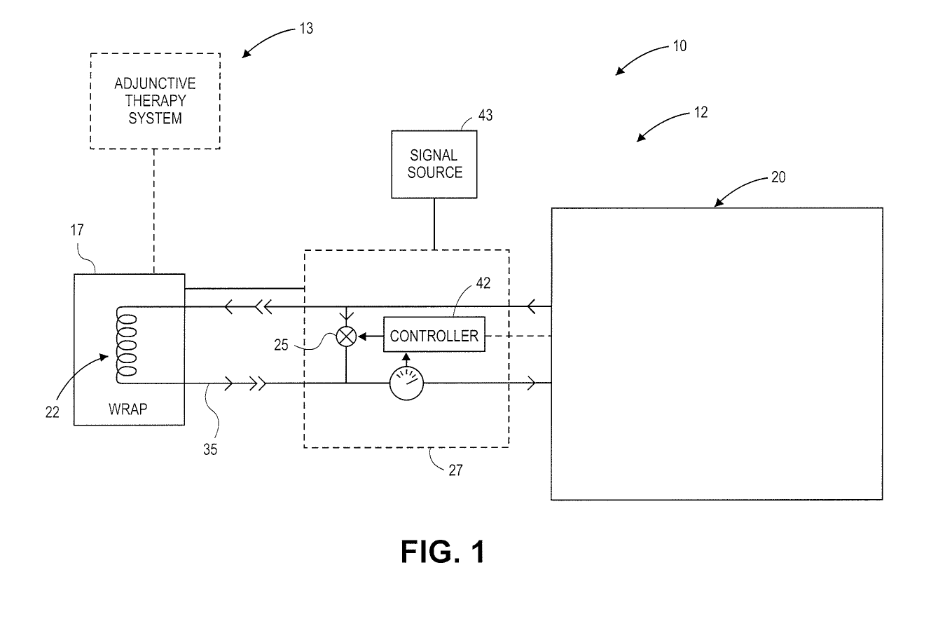

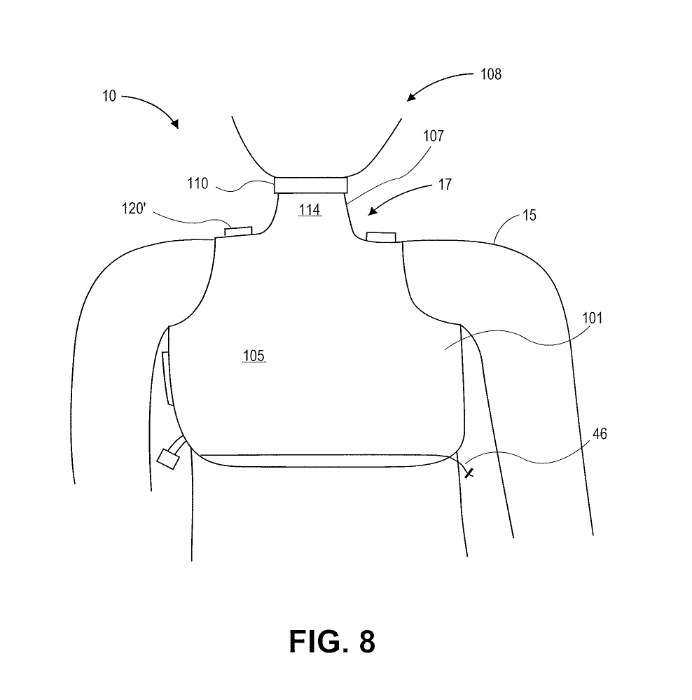

FIG. 1 illustrates an embodiment of a simplified therapy system 10. The exemplary therapy system comprises a first therapy device, generally designated 12, and a second therapy device, generally designated 13. In various embodiments, the first therapy device is a thermal therapy device for delivering temperature-controlled therapy to an animate body 15 (shown in FIG. 8). The second therapy device, described in greater detail below, may include any of a number of devices for treating a body in need of treatment as would be understood by one of skill in the art. System 10 includes a therapy wrap 17 for applying to the animate body.

Wrap 17 is a cover for wrapping around at least a portion of animate body 15. The exemplary wrap is in the form of a sleeve that surrounds or clasps to a portion of the body.

The exemplary wrap is adapted to connect the first and second therapy devices to the animate body in coordination with each other. By coordination it is meant that the first and second therapy devices do not physically interfere with each other or that they can be connected to the body at the same or nearly the same time. In various respects, coordination refers to the fact that each of the therapy devices does not substantially interfere with the therapy to be applied by the other. Because patients often need to receive a variety of treatments at once, the system described herein thus has the advantage of allowing multiple treatments to be applied in close time without reduced interference. In the case of cardiac arrest, for example, the body may be cooled at or near the same time that a defibrillator is applied to the body. With conventional techniques, by contrast, it can be difficult or impossible to apply different treatments simultaneously or in quick succession. Moreover, many devices such as defibrillators are difficult to attach to the body at the same time as existing thermal therapy devices.

Many medical and therapeutic devices may be used within the scope of the invention. The second therapy device may include, but is not limited to, a defibrillator, a CPR device, a neuromuscular electric stimulation device, a sensor or monitoring device (e.g. EKG), a blood pressure cuff, an intravenous (IV) tube, a brace (e.g. joint brace), airway/ventilation equipment, and transport structures (e.g. backboards, stretchers, restraints, etc.). In various embodiments, the second therapy devices includes at least one the following a defibrillator, a heart monitor, a CPR device, and a neuromuscular electric stimulation device.

In various embodiments, system 10 comprises a therapy wrap 17 and first therapy device 12. Instead of a second therapy device with working elements and the like, the system is configured for permitting administration of a treatment or surgery in conjunction with thermal therapy. For example, therapy wrap 17 may be configured to provide core body cooling while allowing access by a physician for surgery. The surgery may be open surgery or less invasive procedures.

The exemplary system has a modular layout. The first modular member includes therapy wrap 17. Various modular members may be inserted into or attached to the therapy wrap. For example, a bladder assembly of a heat transfer device may be inserted into a pouch of the wrap. Similarly, other elements may be attached to therapy wrap 17 alone or in combination with the heat transfer device. The other system components such as the control elements, cooling source, power source, and monitor may be provided in a modular configuration as would be understood by one of skill from the description herein.

The modular members may be readily removed from therapy wrap 17 so that the wrap configuration can be quickly and easily changed. This also allows the various constituent modular members to be cleaned or replaced more easily. In various embodiments the therapy wrap is disposable. This may be helpful, for example, in medical applications where it is difficult to sterilize the wrap for reuse. This may be helpful in less regulated environments as well such as in a sports clinic where the wrap may be stained with blood, sweat, and other fluids. The modular configuration allows the bladder assembly protected inside to be replaced, as necessary, independently from the wrap.

In another example system 10 includes working electrodes for applying electric stimulation to a patient. The modular configuration described above allows the electrodes to be maintained and replaced in an otherwise conventional manner. For example, a protective layer may be applied to the electrode surfaces so they can be reused. Thus, wrap 17 can be disposed and the electrodes can be reused. The ability to replace one modular member can thus avoid the need to dispose of the entire apparatus, thereby providing the ability to reduce cost over time.

In various embodiments, therapy wrap 17 is configured to be cleaned. For example, the wrap may be formed of materials that are easy to clean with conventional cleaning processes. Such materials include non-porous fabrics and flexible plastics. The wrap may be formed of different materials such that the areas that are likely to be contaminated are removable for disposal or cleaning. For example, wrap 17 may include a frame structure comprising straps for hanging the wrap from on the body. The chest area or other areas at risk of contamination may be formed as separate members and secured to the frame structure. In other example, the wrap is configured so that certain portions may be cut out and replaced.

The exemplary first therapy device 12 and second therapy device 13 are independently connected to therapy wrap 17. One will appreciate that the first and second therapy devices may be connected to the wrap in various ways such as serially, in parallel, and the like. Various components of the therapy devices may be connected to a single control unit that is connected to the therapy wrap. For example, the working components of the therapy devices may be connected to the wrap while the other components of the therapy devices are connected to a control unit or other device.

The modular configuration described herein also allows for improved interchange of parts. In various embodiments, a set of therapy wraps may be provided to a user. The user can then select one of the wraps based on particular characteristics (e.g. patient size, patient weight, indication, desired treatment). The necessary working elements can then be attached to the selected wrap for use. The description herein will readily make apparent many other advantages of the invention.

### II) Thermal Therapy Device

Various aspects of the invention are similar to the subject matter described in: U.S. patent application Ser. No. 09/127,256 (filed Jul. 31, 1998) entitled, “Compliant Heat Exchange Panel,” issued on Apr. 3, 2007 as U.S. Pat. No. 7,198,093; U.S. patent application Ser. No. 09/798,261 (filed Mar. 1, 2001) entitled, “Shoulder Conformal Therapy Component of an Animate Body Heat Exchanger,” published on Aug. 30, 2001 as U.S. Publication No. 2001-0018604A1; U.S. patent application Ser. No. 09/901,963 (filed Jul. 10, 2001) entitled, “Compliant Heat Exchange Splint and Control Unit,” published on Nov. 8, 2001 as U.S. Publication No. 2001-0039439A1; U.S. patent application Ser. No. 09/771,123 (filed Jan. 26, 2001) entitled, “Wrist/Hand Conformal Therapy Component of an Animate Body Heat Exchanger,” published on Oct. 25, 2001 as U.S. Publication No. 2001-0034546A1; U.S. patent application Ser. No. 09/771,124 (filed Jan. 26, 2001) entitled, “Foot/Ankle Conformal Therapy Component of an Animate Body Heat Exchanger,” published on Feb. 14, 2002 as U.S. Publication No. 2002-0019657A1; U.S. patent application Ser. No. 09/771,125 (filed Jan. 26, 2001) entitled, “Conformal Therapy Component of an Animate Body Heat Exchanger having Adjustable Length Tongue,” published on Oct. 25, 2001 as U.S. Publication No. 2001-0034545A1; U.S. patent application Ser. No. 10/784,489 (filed Feb. 23, 2004) entitled, “Therapy Component of an Animate Body Heat Exchanger,” published on Aug. 26, 2004 as U.S. Publication No. 2004-0167594A1 which is a continuation of U.S. patent application Ser. No. 09/765,082 (filed Jan. 16, 2001) entitled, “Therapy Component of an Animate Body Heat Exchanger and Method of Manufacturing such a Component,” issued on Feb. 24, 2004 as U.S. Pat. No. 6,695,872 which is a continuation-in-part of U.S. patent application Ser. No. 09/493,746 (filed Jan. 28, 2000) entitled, “Cap And Vest Garment Components Of An Animate Body Heat Exchanger,” issued on Jan. 30, 2001 as U.S. Pat. No. 6,178,562; U.S. patent application Ser. No. 10/122,469 (filed Apr. 12, 2002) entitled, “Make-Break Connector For Heat Exchanger,” issued on Mar. 29, 2005 as U.S. Pat. No. 6,871,878; U.S. patent application Ser. No. 10/637,719 (filed Aug. 8, 2003) entitled, “Apparel Including a Heat Exchanger,” issued on Sep. 19, 2006 as U.S. Pat. No. 7,107,629; U.S. patent application Ser. No. 12/208,240 (filed Sep. 10, 2008) entitled, “Modular Apparatus for Therapy of an Animate Body,” published on Jan. 1, 2009 as U.S. Publication No. 2009-0005841A1 which is a divisional of U.S. patent application Ser. No. 10/848,097 (filed May 17, 2004) entitled, “Modular Apparatus for Therapy of an Animate Body,” issued on Mar. 1, 2011 as U.S. Pat. No. 7,896,910; U.S. patent application Ser. No. 11/707,419 (filed Feb. 13, 2007) entitled, “Flexible Joint Wrap,” issued on Nov. 23, 2010 as U.S. Pat. No. 7,837,638; U.S. patent application Ser. No. 11/854,352 (filed Sep. 12, 2007) entitled, “Make-Break Connector Assembly with Opposing Latches,” issued on Jun. 8, 2010 as U.S. Pat. No. 7,731,244, which is incorporated herein for all purposes by reference.

The above systems generally provide active heating, cooling, and/or compression for humans and other animal bodies. They are used, for example, in physical therapy, pre-game conditioning, minor injury care, post-operative care, and emergency medical care, among other applications. Thermal therapy systems exist in a number of different forms. In general, there is a control unit, a connector hose, wrap comprising a heat transfer device and cover, and a power source (i.e., battery or external electric power). The therapy wrap comprising the cover and the heat exchanger are applied to the portion of the mammal's body to receive therapy. The control unit is used to modulate a heat transfer medium in the wrap to achieve the desired therapeutic result. One such system is disclosed, for example, in U.S. Pat. No. 6,178,562, the disclosure of which is herein incorporated by reference.

In various embodiments, first therapy device 12 is a thermal therapy device for exchanging heat with body 15 via a heat transfer medium. The exemplary thermal therapy device is configured for applying cold therapy to a body.

FIGS. 1-4 illustrate a representative number and type of components used in an exemplary cold therapy system. The therapy device includes a cooling source 20, a heat transfer device 22, a pump 25, and a control unit 27. Exemplary heat transfer device 22 is in the form of a multi-bladder assembly for positioning adjacent the body surface at the treatment site and circulating the heat transfer medium. The control unit controls the flow of fluids to the heat transfer device. The arrows in FIG. 1 indicate the fluid flow exiting/leaving cooling source 20 into wrap 17 as well as leaving the wrap and entering the cooling source. In general, the body exchanges thermal energy with the heat transfer medium when the fluid is flowing. Control unit 27 may also be configured for other functions such as displaying the system status and programming the treatment parameters as will be described in greater detail below. In the exemplary embodiment, cooling source 20 is housed within control unit 27.

Exemplary cooling source 20 comprises a cold water reservoir. Control unit 27 is connected to therapy wrap 17 using a connector hose 35. Pump 25 is in communication with reservoir 20 and wrap 17. The exemplary control unit includes a central processing unit (CPU), input/output components, and user adjustment controls. Under control of the control system within control unit 27, the pump takes water from the reservoir and circulates it through heat transfer device 22 in wrap 17, after which it returns to the reservoir.

A commonly used external thermal bladder assembly uses both a compliant fluid bladder for circulating heat transfer fluid and a gas pressure bladder which overlays the fluid bladder. The gas pressure bladder is adapted to inhibit edema and/or for pressing the fluid bladder against the body part to be subjected to heat exchange. In general, the body heat exchanging component(s) of such an apparatus has a pair of layers defining a flexible bladder through which a liquid is circulated. These heat exchanging components are sometimes referred to herein as the “heat transfer device.”

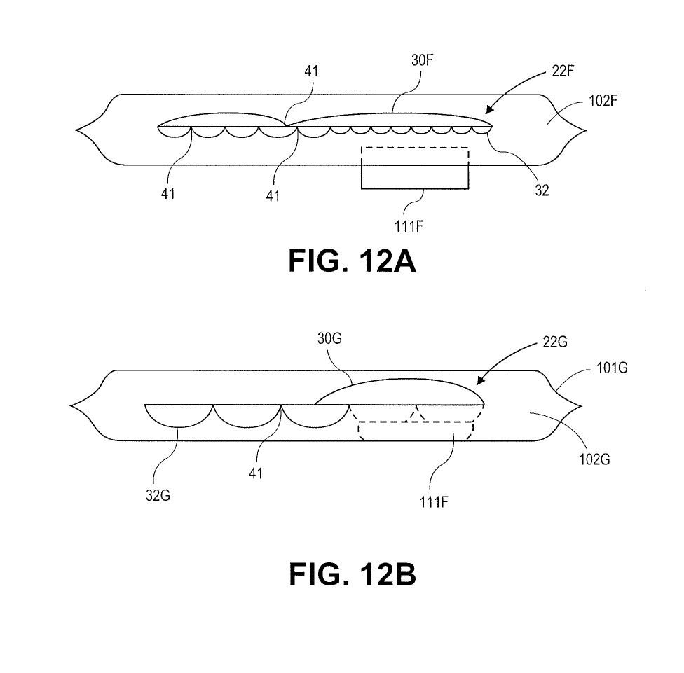

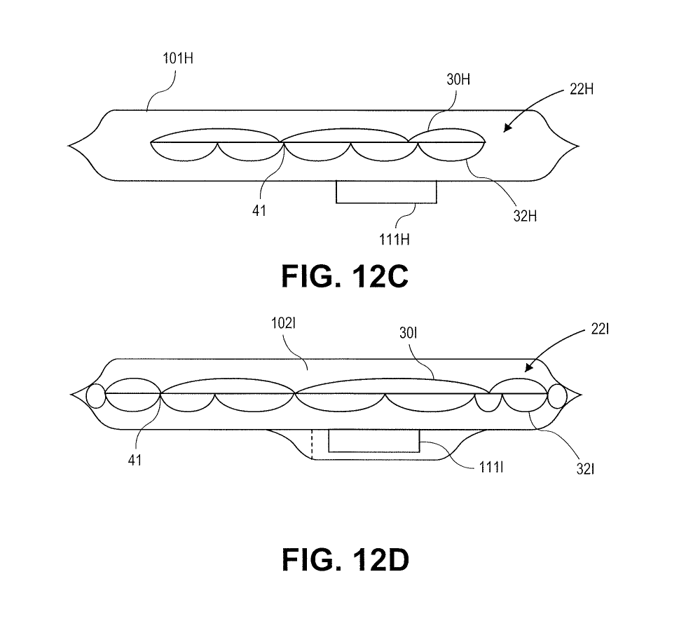

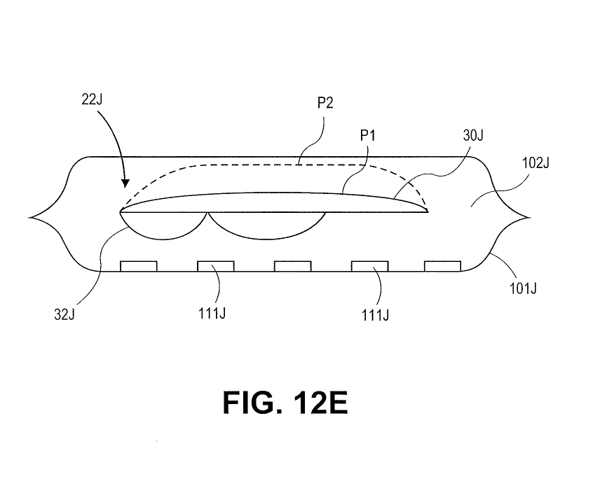

Heat transfer device 22 in therapy wrap 17 is shown in the treatment position adjacent body 15 in FIG. 8. Heat transfer device 22 includes at least one body heat exchanging component. Exemplary heat transfer device 22 comprises at least two compliant bladders: outer bladder 30 and inner bladder 32 (shown in FIG. 12A-12F). The exemplary outer bladder is an expandable gas pressure bladder for applying a compressive force. The exemplary inner bladder is a fluid bladder for circulating the heat transfer medium.

More specifically, outer bladder 30 is adapted to receive a first fluid such as a gas (e.g., air), which can be regulated to provide the desired amount of inflation of the bladder or pressure therein. This inflation or pressure affects the compressive force applied to the animate body during use.

Inner bladder 32 is adapted to receive a fluid, such as a coolant, which can be in the form of a cold liquid, to transfer heat away from the animate body part. Alternatively, the fluid supplied to the inner bladder can have a temperature higher than the animate body part to heat the body part.

Additional bladders and elements may be provided between the inner and outer bladder. For example, one or more intermediate bladders configured to adjust the rigidity of the wrap may be sandwiched between the inner and outer bladders. It may be desirable to reinforce one or both of the inner and outer bladder in a kink-prone region using a structural reinforcement or adjusting a weld pattern such as disclosed in U.S. patent application Ser. No. 12/939,986 to Lowe, the entire contents of which are incorporated herein for all purposes. In various embodiments, therapy wrap 17 and/or heat transfer device 12 include a rigid member in key cooling regions such as the chest region and neck region to reduce the risk of obstruction of flow. In various embodiments, the rigid member is preformed to the shape of the part of the body to which it is intended to be applied.

The exemplary fluid bladder 32 includes a pair of layers of flexible material sealed together to form a fluid-tight chamber. Compliant gas pressure bladder may also be defined by a pair of generally parallel and flexible layers of material. The bladders form separate chambers for different fluids and are made to preclude fluid communication between the chambers during use.

In various embodiments, bladders 32 and 30 can be formed from three sheets of material with one sheet forming a common inner wall. One side of the common wall aids in defining gas pressure bladder 30 whereas the other side aids in defining fluid bladder 32. Thus, three compliant walls or sheets of material are all that is necessary to define the two separate bladder chambers. The inner wall is also secured to the outer walls along its perimeter. The securing can be performed by RF welding.

In various embodiments, the sheets are nylon material coated with polyurethane to provide both the RF welding qualities and the needed liquid or air impermeability. In one embodiment of the invention, the inner surface of the outer wall of the fluid bladder has an extra heavy coating, which corresponds to about a 5 oz coating of polyurethane, while the inner surfaces of the other walls have standard coatings corresponding to about 3 oz coatings of polyurethane. This construction has been shown to produce an extremely robust weld. A finish on the nylon material can also provide a permanent antimicrobial finish to prevent mold growth.

In various embodiments, the fluid bladder includes a plurality of connections, generally designated 41, interiorly of the perimeter as disclosed by U.S. Pat. No. 6,695,872 to Elkins, the entire contents of which is incorporated herein for all purposes by reference. The interior connections may comprise dot connections (spot welds) and linear and/or curvilinear connections (fences). The fences may be positioned and configured to define a fluid flowpath in bladder 32. The dot connections may be positioned and configured to separate the flowpath into smaller fluidic channels. The matrix of connections acts to disperse the fluid throughout the bladder. The dispersion of fluid may be further aided by forming the fences in a curvilinear shape.

The dot connections can be formed in various patterns and configuration as described in U.S. patent application Ser. No. 12/939,986 to Lowe, the entire contents of which are incorporated for all purposes by reference. In some regions the dots may have a relatively uniform distribution. The dots may be formed in a triangular grid. In other regions, such as a region prone to kinking, the dots may be spaced further apart and formed in a random arrangement. The dots may also have a larger diameter to increase the resistance to kinking.

The connections may also serve to control inflation of the bladder and reduce “ballooning.” In various embodiments, connections are formed in gas pressure bladder 30. Connections can be formed in the inner and outer bladders by forming some of the connections through all three sheets. The result is that these connections are formed in gas bladder 30 and fluid bladder 32. It appears functionally as if the desired connections provided in the liquid bladder are “telegraphed” to appear in the gas bladder. These connections in the two bladders register with one another. Alternatively, the connections in each bladder may be varied by first forming some connections between the first two sheets of material (i.e. the inner and middle sheets), overlaying the outer sheet, and then forming additional connections through all three sheets.

In the illustrative embodiment, the shape of gas pressure bladder 30 conforms to the shape of fluid bladder 32. Fences or dividers in the heat exchange bladder to direct fluid flow can be also provided in the gas pressure bladder not only for directing the flow of a liquid or gas but also to secure the walls defining the gas pressure bladder together at various locations within the interior of such bladder.

With reference to FIGS. 1, 2, and 3, the heat transfer device control unit and cooling function will now be briefly described. In various embodiments, the heat transfer fluid fed to the heat transfer device is maintained at a desired temperature. Generally, the desired temperature is lower or higher than the temperature expected for the body part. In a typical cold therapy system, the heat transfer fluid is cooled prior to the inlet to the fluid bladder by passing the fluid through a heat exchanging medium such as cooling source 20. One such system is disclosed, for example, in U.S. Pat. No. 6,178,562, the disclosure of which is herein incorporated for all purposes by reference.

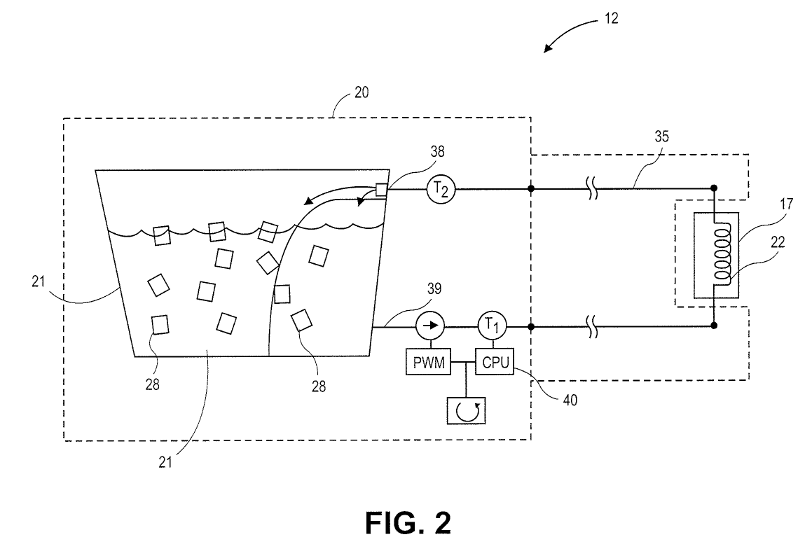

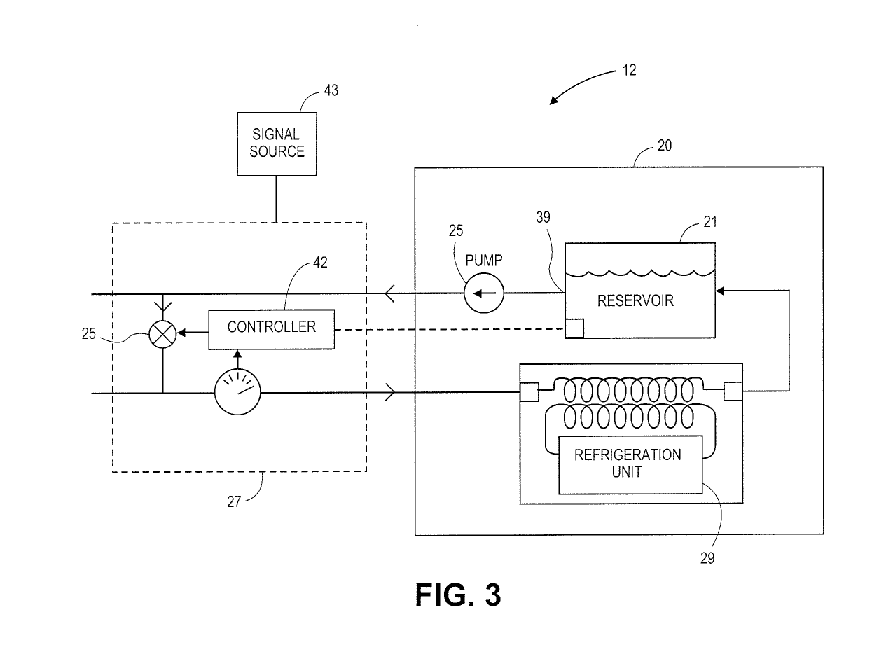

The exemplary cooling source 20 is a reservoir 21 physically housed within control unit 27 and containing a liquid bath. FIG. 2 illustrates cooling of the reservoir by inserting cold elements such as ice. FIG. 3 illustrates cooling of the reservoir with by circulating the feed liquid through a refrigeration unit in accordance with one embodiment. In a practical realization of these embodiments, the liquid is normal tap water. In various embodiments, temperature in the reservoir is in a range between 40° F. and 50° F.

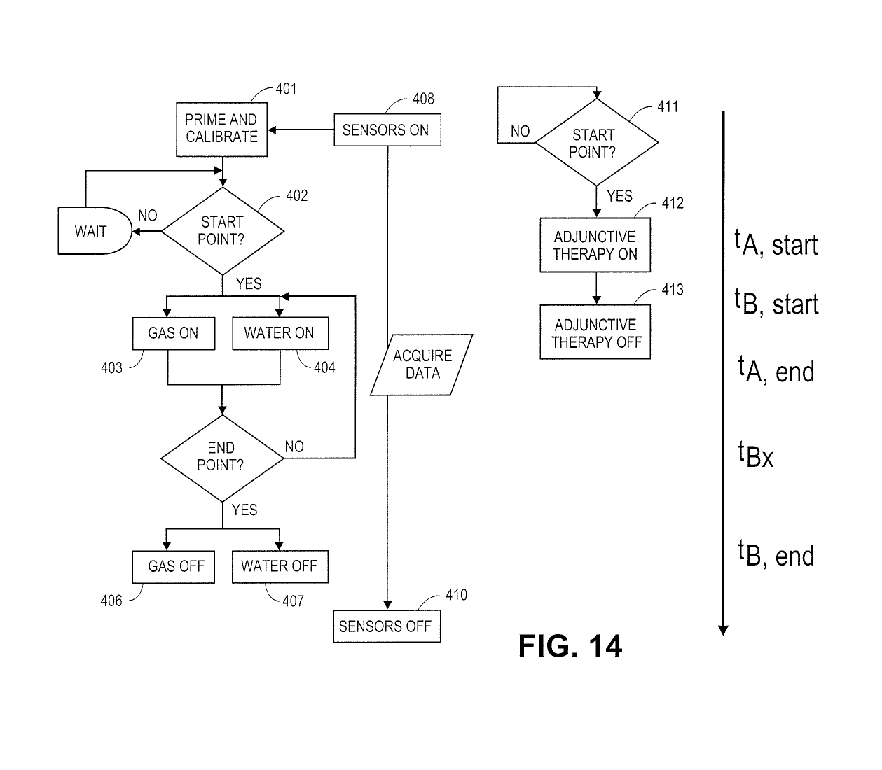

In the exemplary system of FIG. 2, the liquid is cooled by placing ice 28 into an ice box portion of control unit 27. In this connection, control unit 27 accepts liquid that has been returned from heat transfer device 22. Before reintroducing the heat exchange liquid into heat transfer device 22, it can be mixed with the liquid in reservoir 21 or it can be directed to bypass the reservoir. That is, the control unit is capable of supplying liquid at other controlled temperatures by means of mixing liquid chilled in the cooling source with returned liquid warmed in the heat transfer device by contact with an animate body. Other cooling sources may be provided for lowering the heat transfer device directly or indirectly as would be understood by one of skill from the description herein. Examples of other cooling sources will be described below with reference to FIGS. 14A, 14B, and 14C.

The system of FIG. 3 is similar to FIG. 2 except that the liquid is cooled by a refrigeration unit 29. The refrigeration unit is positioned serially with the reservoir 21 such that feed liquid to the reservoir is cooled by the refrigeration unit. The exemplary refrigeration unit is a conventional device whereby the fluid is cooled as it winds through a fluid channel in a heat exchanger. The refrigeration unit may provide faster and more sustained cooling of the reservoir. The fluid begins to be cooled as soon as the refrigeration unit is turned on such that cooled fluid can be delivered to therapy wrap 17 near instantaneously. The reservoir will then reach the desired temperature nearly as fast as it can be supplied with fluid from the refrigeration unit. This can be advantageous in situations where cooling is needed quickly such as emergency medical services. This can also be advantageous in applications where it is inconvenient to store or obtain ice.

Exemplary reservoir 21 is a container with an interior defined by a floor and walls. There is an inlet 38 in fluid communication with the interior and heat transfer device 22 positioned in therapy wrap 17. In an exemplary embodiment, an outlet 39 fluidly communicates with the interior through a penetration in a wall of the container at a location closer to the floor than inlet 38. The outlet is in fluid communication with pump 25. In some embodiments, inlet 38 is above the surface of the heat transfer fluid when in use. When in use the heat transfer fluid exiting the inlet enters the interior above the surface of the heat transfer fluid within the container.

As shown in FIG. 2, the inlet may be moved to adjust the flow of fluid across the reservoir. The inlet location may be controlled by a pulse width modulation (PWM) controller and central processing unit (CPU). Further details regarding the inlet adjustment mechanism may be found in U.S. patent application Ser. No. 12/910,772 to Lowe et al., incorporated herein for all purposes by reference.

Performance of thermal therapy device 12 may be improved by adjusting the heat transfer fluid flow rate, adjusting the heat transfer device temperature, and/or providing additional features to the thermal therapy device. In a return flow arrangement such as that shown in FIG. 1, by example, the velocity of the fluid and heat transfer rate are generally proportional to the flow rate. Reducing the flow rate of the fluid of a given temperature through heat transfer device 22 will also reduce the amount of energy removed from (or added to) the patient. Conversely, increasing the flow rate will increase the amount of energy removed from (or added to) a patient. In a cold therapy device, with the wrap applied to a mammalian body, the temperature of the fluid leaving the wrap is warmer than the temperature of the fluid entering the wrap because the mammalian body is typically warmer than the thermal fluid.

As the fluid flow rate into the wrap becomes slower, the temperature delta increases as does the average wrap temperature. To decrease the average wrap temperature, the flow may be increased sufficiently. A slower flow rate, however, may lead to less efficient heat transfer and other performance problems.

Lowering the temperature from reservoir 21, which determines the inlet temperature of heat transfer device 22, generally leads to a lower average heat transfer device temperature and increased heat transfer. For example, if an average wrap temperature of 5° C. is desired, then an inlet temperature of 1° C. may be needed. In this example, the temperature delta across the heat transfer device may be 10° C., which is quite large. Moreover, the inlet temperature is near freezing. For use with humans, this may be uncomfortable at best and, at worst, cause cold burns during extended periods of use. In various embodiments, thermal therapy device 12 is configured to reduce patient discomfort. Examples of techniques for achieving desired heat transfer with reduced patient discomfort are disclosed by U.S. patent application Ser. Nos. 12/910,772 and 12/910,743 (attorney matter nos. 11185-700.200 and 11185-700.201), the entire contents of which are incorporated herein by reference.

The control unit for the thermal therapy device in accordance with the invention will now be described in greater detail. FIGS. 1-4 illustrates an exemplary embodiment of control unit 27 and representative components of thermal therapy device 12 connected to the control unit. Exemplary control unit 27 is fluidly connected to heat transfer device 22 as explained above. The exemplary control unit is configured to regulate compressive pressure and fluid to the heat transfer device.

Exemplary control unit 27 for thermal therapy device 12 includes a processor 40, such as a central processing unit (CPU) or microprocessor and memory. The control unit may also include a controller 42 for generating a control signal. The memory includes random-access memory such as a dynamic random access memory (DRAM) or static random access memory (SRAM), and nonvolatile memory such as an electrically erasable programmable read-only memory (EEPROM). The EEPROM can be used to store software programs executed by the microprocessor to control operation of the device, as will be described in detail below.

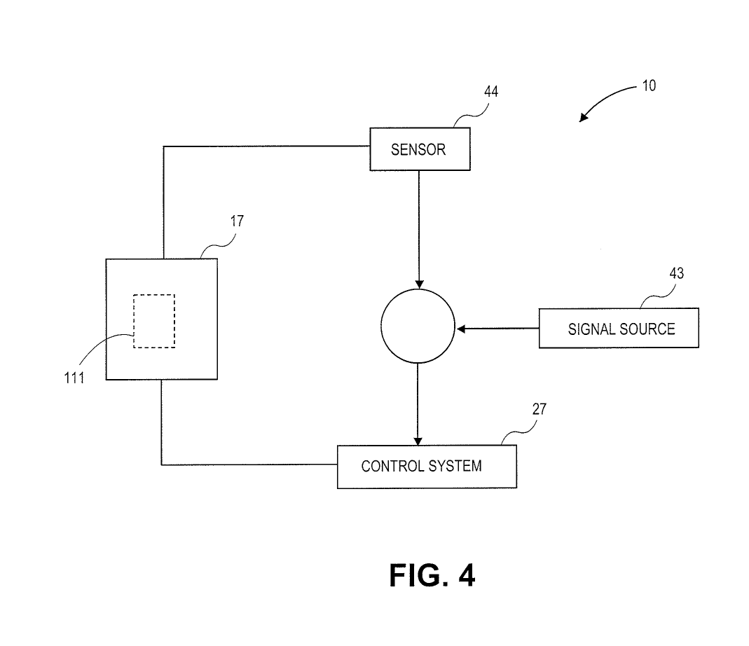

FIG. 4 is a block diagram illustrating the basic flow of data in system 10. The exemplary system of FIG. 4 is the basic set up for thermal therapy device 12. One will appreciate however, that the principles described can apply equally to first therapy device, second therapy device, or both.

FIG. 4 illustrates representative components of system 10 connected to therapy wrap 17. System 10 includes control system 27 for receiving an input signal and outputting a control signal. The control system adjusts operation of the therapy wrap based on the output control signal. The input signal is generated by sensors 44 and/or a signal source 43. The signal source information may be derived from a number of sources. The signal source information may include, but is not limited to, feedback and monitoring information from the wrap and the control system.

In various embodiments, control unit 27 receives and processes input information. The exemplary system includes one or more sensors 44 that communicate with the control unit. The sensors may be attached to selected components as would be understood by one of skill in the art. The sensors may be configured to monitor characteristics of the system components or the body. The sensor information may then be used by control unit 27 to operate components of system 10 to produce the desired therapeutic result.

As shown in FIGS. 1, 2, and 4, the control unit may be configured to regulate the return system. The return system may be a conduit used to return the heat transfer fluid in the system back to the reservoir 21. Additionally or alternatively, the return system may include valves, diverters or other flow control elements (not shown). The reservoir may include one or more reservoir inlets or reservoir outlets, a baffle, a filter, a diffuser or any of the reservoir improvements.

In various embodiments, the system acquires data from sensors and the information is processed for diagnostic or troubleshooting functions. For example, the system may determine that there is a leak or a blockage (e.g. a kink in the fluid pathway) if the fluid flow rate drops below a predetermined threshold. The system may determine that there is a blockage if the backpressure rises above a predetermined threshold.

The input information to the controller can also be used to derive information about system performance and patient response. The information may be fed back into the system to optimize performance. Information that can be input to the control unit includes, but is not limited to, heat transfer device inlet temperature, heat transfer device outlet temperature, cooling source inlet temperature, cooling source outlet temperature, heat transfer fluid flow rate, elapsed time, and combinations of the same. Sensors may also be selectively positioned on the body to measure or estimate core temperature.

In various embodiments, control unit 27 includes input controls, an output device, and input/output (I/O) circuitry. The exemplary control unit includes switches to allow a user to input information such as patient indication (condition). The control unit may also include a communications port for receiving information. An output device (not shown) such as a digital display or illuminated lights can provide information to the user.

In various embodiments, control unit 27 receives information related to other therapies. For example, second therapy 13 may send start and stop signals to the control unit to indicate when the cooling should be adjusted based on starting of another therapy device connected to the system. The control unit may receive any of the above information as inputs, process the input information, and make a determination. For example, the control unit may determine whether and how to adjust the function of first therapy device 12.

Although system 10 thus far has been described in terms of having independent control units for first therapy 12 and second therapy device 13, one will appreciate than many of the functions and devices may be combined in different combination. In various embodiments, system 10 includes a single control unit and processor. In an exemplary embodiment where the system includes an electrical stimulation device and thermal therapy device, for example, the thermal therapy device does not need a large power source and complicated circuitry. Thus, the processing and control function of the thermal therapy device could be easily integrated into the control unit for the electric stimulation device. The cooling source and gas pressure source could be provided separately as would be understood by one of skill from the description herein.

The exemplary heat transfer device includes a compressive mechanism, namely, compressive bladder 30. Control unit 27 is also fluidly connected to the compressive bladder 30.

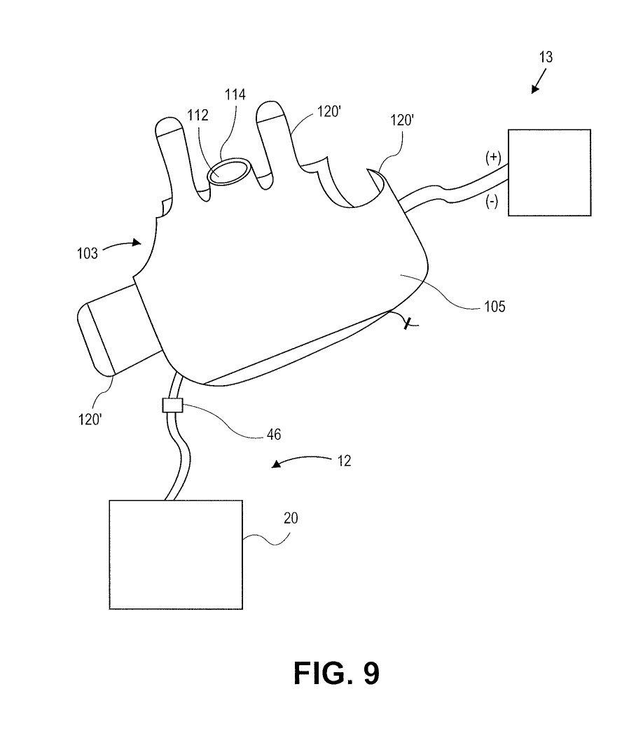

In various embodiments, a single connector 46 is provided for connecting the heat transfer medium—the compressive bladder and fluid bladder—to the control unit (shown in FIG. 9). U.S. Pat. No. 6,871,878, incorporated herein for all purposes by reference, discloses a three-port manifold connector that may be used with the present system. The three-port manifold includes a port for a gas to be introduced and exhausted from gas pressure bladder 30 and fluid inlet and outlet ports for circulating fluid through fluid bladder 32. The ports typically have an inner diameter of about ⅛-inch. The manifold passageways typically have a diameter of about ¼-inch. Other suitable manifold constructions are disclosed in U.S. Pat. Nos. 5,104,158 and 5,052,725, both to Meyer et al. and both hereby incorporated herein for all purposes by reference.

Each of the manifold fluid inlet and fluid outlet passageways may be provided with a valve, such as a spring loaded valve, to allow the selective passage of fluid therethrough. The valves may allow flow when the fluid hose connectors are coupled to the manifold and prevent fluid flow when the fluid hose connectors are uncoupled from the manifold as would be understood by one of skill from the description herein. In this manner, fluid such as a liquid coolant is blocked from exiting fluid bladder 32 when the fluid hoses are uncoupled from the manifold.

The manifold, which carries or forms the tubular members, can be configured to mate with the curves of the body when connected to the modular apparatus. It also can be provided with a ridge for finger placement to allow easier removal. It should be understood that other manifold configurations and/or couplings to provide fluid flow between the fluid source and the bladders can be used as would be apparent to one of skill in the art. For example, valves need not be provided in the liquid port couplings. The valves may also be controlled by an actuator and the control unit. For example, the control unit may control the fluid flow by opening and closing the valves based on control parameters.

In addition to controlling heat transfer by the flow of heat exchange medium to the fluid bladder 32, control unit 27 also controls the flow rate and pressure of gas supplied to the compressive bladder 30 to control inflation, deflation, and compressive pressure. In various embodiments, the gas delivered to gas pressure bladder 30 is compressed air. In various embodiments, the pressure of gas furnished by the control unit is between about 0.25 psig and about 20 psig, preferably between about 0.25 psig and about 5 psig, and more preferably about 0.25 to about 1.5 psig.

In various embodiments, system 10 may be controlled manually by a user or automatically. The system may be configured to operate based on and off modes. Alternatively, the system may utilize control unit 27 to monitor and/or regulate fluid flow through pump 25 and therapy wrap 17.

### III) Supplemental Therapy Device

Referring to FIGS. 1, 4, 5, and 6, system 10 comprises second therapy device 13, generally referred to below as a supplementary therapy device. Supplementary device 13 supplements, replaces, and/or augments the thermal therapy and/or compression therapy. In the exemplary second therapy device 13 works in conjunction with first therapy device 12.

The exemplary second therapy device is an electric stimulation. Although exemplary supplemental therapy device is described in terms of an electric stimulation device, one will appreciate that a variety of other therapy devices may be used with the system in accordance with the invention. Suitable devices may include electrical, chemical, and mechanical working elements. Devices and systems that may be incorporated into the system of the invention include, but are not limited to, a device for delivering energy or a drug, a stabilization device, and a monitoring device. In various embodiments, the energy delivered by the device is electrical energy, microwave energy, radiation energy, energy appropriate for optical or light-based therapy such as ultraviolet, low level laser or visible light energy, and/or infrared energy. A suitable light energy device may be a light emitting diode (LED). In various embodiments, the energy delivered by the device is electrical energy. The electrical energy delivery device may be a defibrillator, ablation device, cardiac pacing device, neuromuscular electric stimulation device, and more. In various embodiments, the system includes a defibrillator, a CPR device, a neuromuscular electric stimulation device, a sensor or monitoring device, a blood pressure cuff, an intravenous therapy device, a brace, and a combination of the same. In various embodiments, system 10 includes two or more second (supplementary) therapy devices.

In various embodiments, the supplemental therapy device is a cardiopulmonary resuscitation (CPR) device. An exemplar of a CPR device that may be used with the therapy wrap and sleeve of the invention is disclosed in U.S. Pat. No. 5,634,886 to Bennett et al. and U.S. Pat. No. 6,616,620 to Sherman et al, the entire contents of which are incorporated herein for all purposes by reference. For example, a compression device may be provided on an inside of therapy wrap 17 for applying compression to the chest. One of skill will also appreciate from the description herein a number of features and accessories that may also be incorporated into the system to aid in the administration of the thermal and supplemental therapies.

In an exemplary embodiment, second therapy device 13 of system 10 is configured to deliver a high energy electrical pulse. Various aspects of the exemplary electrical stimulation device, including the internal hardware, are similar to those in a LIFEPAK® 500 AED available from Medtronic Physio-Control Corp. of Redmond, Wash., the ALS R Series® available Zoll Medical Corp. of Chelmsford, Mass., and the Forerunner® defibrillator available from Heartstream of Seattle, Wash. Other exemplary electrical stimulation devices are disclosed by U.S. Pat. No. 7,797,044 to Covey et al.; U.S. Pat. No. 6,961,611 to Dupelle; and U.S. Pat. No. 5,593,426 to Morgan et al., the entire contents of which are incorporated herein for all purposes by reference. The '044 patent discloses a portable defibrillator.

One of the most common and life-threatening medical conditions is ventricular fibrillation, a condition where the human heart is unable to pump the volume of blood required by the human body. There are generally four critical components of medical treatment that must be administered to a victim of sudden cardiac arrest: (1) early access to emergency care; (2) early cardiopulmonary resuscitation to keep the blood oxygenated and flowing to the victim's brain and other vital organs; (3) early defibrillation (the application of a strong electrical shock to the heart) to restore the heart's regular rhythm; and (4) early access to advanced medical care.

When a person is experiencing sudden cardiac arrest, the electrical activity within the heart becomes chaotic. An electric shock from a defibrillator can reorganize the electrical impulses to allow coordinated pumping action to resume. To administer this shock, special pads from a machine called a defibrillator are placed on the victim's chest, and an electric shock is sent through the victim's body from one pad to another.

Defibrillation is a now a widely accepted technique for restoring a normal rhythm to a heart experiencing ventricular fibrillation. External cardiac defibrillators have been successfully used for many years in hospitals by doctors and nurses, and in the field by emergency treatment personnel, e.g., paramedics.

It has been found that defibrillation and cardiopulmonary resuscitation delivered promptly (e.g. within about four minutes) can increase the victim's chances of surviving sudden cardiac arrest to nearly forty percent. Prompt administration of defibrillation within the first critical minutes is considered one of the most important components of emergency medical treatment for preventing death from sudden cardiac arrest.

Exemplary system 10 includes many of the components and features of conventional electrical stimulation devices, in particular external defibrillators. In general, cardiac defibrillators operate by storing electrical energy and delivering an electric charge at selected times. The external cardiac defibrillator accumulates a high-energy electric charge in an energy storage capacitor. A switching mechanism switches the system from an accumulation state to a delivery state whereby stored energy is transferred to a patient in the form of a large current pulse.

Defibrillator systems typically use a high-energy transfer relay as the switching mechanism. A discharge control signal causes the relay to complete an electrical circuit between the storage capacitor and a wave shaping circuit whose output is connected to the electrodes attached to the patient. The relay can provide a monophasic or biphasic waveform to the patient.

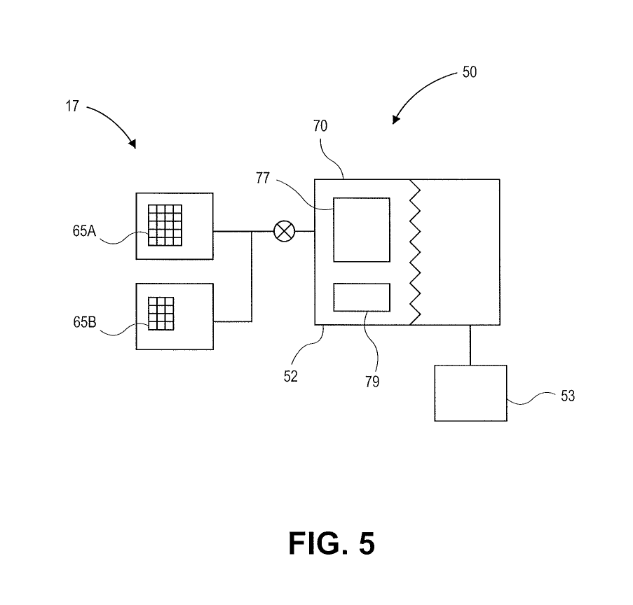

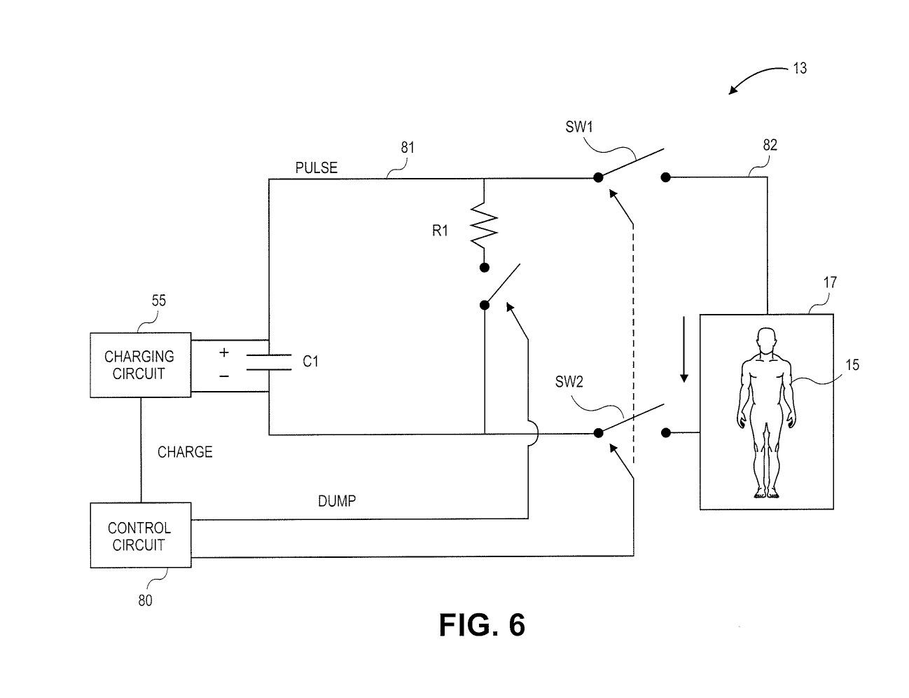

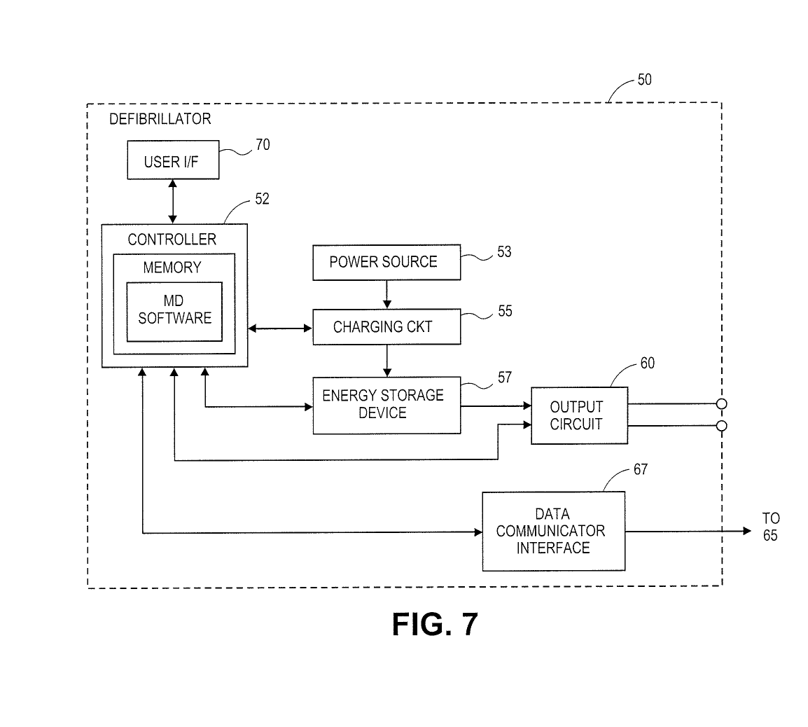

FIGS. 5-7 illustrate an exemplary system 10 including a second therapy device 13 in the form of an electric stimulation device 50, in particular, a defibrillator. Exemplary electric stimulation device 50 is an external portable defibrillator. FIG. 5 illustrates the components of the device. FIG. 6 is a block diagram of the exemplary defibrillator of FIG. 5 illustrating the internal components in greater detail. Although described in terms of a defibrillator, those skilled in the art will be able to implement other embodiments using other types of medical equipment using the principles described herein.

The exemplary defibrillator 50 includes a controller 52, a power source 53, a charging circuit 55, an energy storage device 57, an output circuit 60, output electrodes 65a and 65b, a data communicator interface 67, and a user interface 70. The exemplary controller includes an input/output data communication circuit for communicating information with the user interface.

One or more of the components described in connection with supplementary therapy device 50 may be shared with thermal therapy device 12. In system 10, for example, a single controller, power source, and/or user interface control can be shared by thermal therapy device 12 and one or more supplementary therapy devices 50.

Exemplary power source 53 is implemented with a rechargeable battery. Charging circuit 55 is coupled to power source 53. Energy storage device 57 is coupled to the charging circuit and implemented with a capacitor as will be described in greater detail below with reference to FIG. 8. The exemplary capacitor has a capacitance between about 190 and about 200 μF. Output circuit 60 is coupled to energy storage device 57. In operation, as well known in the art, under the control of controller 52, charging circuit 55 transfers electrical energy from power source 53 to energy storage device 57 and output circuit 60 transfers energy from energy storage device 57 to electrodes 65. Data communicator interface 67 is implemented with a standard data communication port. The user interface is implemented with conventional input/output devices, including, for example, a display, speaker, input keys, a button, an alarm, and/or a microphone.

Conventional electrical stimulation devices apply current to the body through electrodes. Typically the devices include one or more pairs of electrodes to create an electrical potential. Some devices use one or more electrodes and a reference electrode. In any case, the device delivers a current pulse to the patient through one or more electrodes. The exemplary device 50 includes electrodes 65 to be positioned on the patient's chest.

Electrodes 65 are configured similar to electrodes for use with conventional defibrillators. The electrodes comprise a conductive surface for placing in contact with the patient's skin and transferring charge. Unlike conventional defibrillators, the exemplary electrodes have a substantially flat shape. Rather, the electrodes comprise a conductive part of the electrode lead and do not include handles. The exemplary electrodes are configured to be positioned between an inner surface of therapy wrap 17 and body 15. In various embodiments, the electrodes are positioned in a sandwich configuration. Positioning and attachment of the electrodes will be described in greater detail below with reference to FIG. 8.

System controller 52 includes a microprocessor such as, for example, a model 68332 available from Motorola, along with memory 72 and software 74. The software may include readily available medical device software that controls the operation of the medical device. The software may be implemented in various forms such as embedded on the processor or in the memory. The software may also be loaded onto a computer-readable medium such as a CD or flash drive. The memory includes random-access memory such as a DRAM (dynamic random access memory) or SRAM (static random access memory), and nonvolatile memory such as an EEPROM (electrically erasable programmable read-only memory). The EEPROM can be used to store the software programs executed by the microprocessor. In addition, the EEPROM may allow the stored software programs to be remotely updated, for example, by downloading updates through a communications port or the Internet.

Although supplementary device 50 is described in terms of having its own controller 52 and circuitry, one will appreciate that the control circuitry and other features of thermal therapy device 12 may be combined with the supplemental device. In various embodiments, system 10 includes a single control unit.

With specific reference to FIG. 5, exemplary supplementary device 50 has a modular configuration. FIG. 5 illustrates the separate electrodes 65, user interface 70, controller 52, and power source 53. The user interface and controller are housed in detachable units. In general, the user interface and controller are connected during transport. In use, the user interface and controller may be separated. The units may be releasably attached using known techniques, for example, latches or pins.

Exemplary user interface 70 includes a monitor 77 and a control panel 79 for controlling operation of supplementary device 50. The monitor can output information related to operation of the device. A user inputs information and controls the device using control panel 79. The control panel may include knobs, a keyboard, buttons, a touchscreen, and the like. It is to be appreciated that a number of different control interfaces can be used in accordance with the invention. The user interface may also receive signals from signal source 43. As described above, the signal source may include a number of sources including sensors 44 on the body and/or connected to the system components.

FIG. 6 is a simplified schematic view of exemplary defibrillator 50, illustrating operation in a monophasic mode. A host control circuit 80 activates capacitor-charging circuit 55 to charge a storage capacitor C1. The capacitor C1 may be charged by a power source (shown in FIG. 6) or using other techniques known in the art. Once capacitor C1 is charged to a sufficient highly voltage, the device can deliver an electric pulse.

To apply a defibrillation pulse, host control circuit 80 activates a control line to close relay switches SW1 and SW2 in response to a control signal PULSE. Relay switches SW1 and SW2 may be mechanical relays or electrical relays such as solid state switching devices. The relay switches may be a single switch or multiple switches. Once the relay switches are closed, a monophasic defibrillation pulse travels from capacitor C1 to patient 15. The pulse energy travels from the positive terminal of capacitor C1 to a line 81 and then through switch SW1. The pulse then passes through a line 82 to the patient. Finally, the charge pulse passes through switch SW2 to the negative terminal of capacitor C1.

If the electrical charge pulse is not to be delivered to the patient, the charge from capacitor C1 is dumped. In this example, capacitor C1 receives a control signal DUMP. To discharge capacitor C1, host control circuit 80 activates a control signal DUMP to close the switches and short out the remaining energy from the capacitor C1 through a dump resistor R1. Exemplary dump resistor R1 allows for slow discharge of capacitor C1 to prevent damage to the circuit components.

The control signals DUMP and PULSE may be generated based on a user or system control. In various embodiments, the system “times out” after a predetermined time has elapsed. One will appreciate that the time-out may be on a number of parameters. For example, the system may time-out based on the lapse of time from when C1 is fully charged, the time since the previous charge pulse delivery, or with respect to the time of an operation of thermal treatment device 12. In various embodiments, one or more of the control signals are based on selected events. For example, the control signal may be triggered by turning the supplementary therapy device on or off. Likewise, the control signals may be triggered by a predetermined thermal therapy device event.

The defibrillator generally described above applies a monophasic waveform to a patient, but one will appreciate that the defibrillator may also be used to apply a biphasic waveform to the patient using the readily available control signals. This may be useful, for example, in applications where it is desirable to reduce the resulting heart trauma associated with the defibrillation pulse.

### IV) Therapy Wrap and Sleeve for Applying Treatment

FIGS. 8, 9, and 10 illustrate an exemplary wrap 17 including a sleeve 101 and various features for improving therapy delivery. The therapy wrap 17 of system 10 is configured for applying to body 15 and delivering treatment. The exemplary therapy wrap is in the form of a sleeve 101 for connecting various components of heat transfer device 12 and supplemental therapy device(s) 13 to the patient's body. The sleeve is similar in many respects to the sleeve disclosed by U.S. Pat. No. 7,896,910 to Schirrmacher et al. and cover disclosed by U.S. Pat. No. 6,695,872 to Elkins, the entire contents of which patents are incorporated herein for all purposes by reference. The sleeve also includes other features to aid in delivery of treatment to the body.

In various embodiments, therapy wrap 17 is adapted to apply working components of the thermal therapy device and supplementary device(s) to a treatment area of the body. Exemplary sleeve 101 is configured to receive one or more heat transfer devices 22 of thermal therapy device 12. As described above, in various embodiments the heat transfer device includes a heat exchanger such as a fluid bladder for circulating a coolant. The heat transfer device, however, may include other heat transfer mechanisms such as those described below.

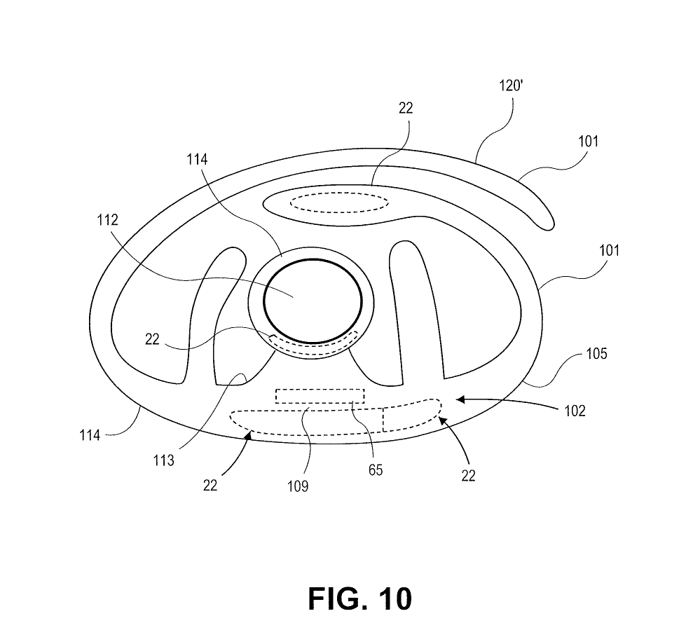

Sleeve 101 has an inner portion 113 and an outer portion 114. The sleeve can be made from various materials and can be formed of inner and outer sheets of material sewn or fused together. For example, the inner and outer sides can comprise two sheets of fabric that are sewn together to form a seam. An additional seam can be provided to form an attachment flap or strap 120 for securing the wrap around body 15. The exemplary sleeve includes a plurality of straps 120. Additional straps 120′ are provided to loop over the shoulders of the body (best seen in FIG. 10).

The inner or outer portion of sleeve 101 may have an opening for directing heat transfer device 22 into a pouch or cavity 102 in the sleeve interior. A portion of sleeve may be pulled back to reveal the pouch and facilitate positioning of the heat transfer device in the pouch. Any suitable fastening means can be used to close the opening such as, but not limited to, a zipper.

Exemplary sleeve 101 also includes a fastener for holding the apparatus in the desired location on the animate body. Accordingly, when the apparatus is wrapped around a portion of or the entire region being treated, the fastener holds the apparatus in place during treatment. In the illustrative embodiment, a hook and loop fastener is used. Referring to FIG. 10, when compression increases, the forces may tend to resolve as shear forces as compared to other forces that can peel the hook portion from the loop portion.

The loop material portion of the hook and loop fastener can be integrally formed with or placed over essentially all of outer (back side) portion of sleeve 101. Alternatively, a strip of loop material can be integrally formed with or placed over a portion or the entire length (measured from the upper to lower edge of the sleeve) of the outer portion along a side opposite attachment flap 120.

The hook material portion of the hook and loop fastener can be in the form of a single strip that extends along the height of inner portion (measured from the upper edge to lower edge of the inner portion), or it can be integrally formed with the inner (front side) portion in the same region. It can extend about 50% to 100% of the length. Alternatively, the hook portion can comprise a plurality of strips which can be spaced along the length of the respective portion of sleeve 101. The exemplary hook and loop fastener is removable such that it can be replaced if it wears out.

In various embodiments, sleeve 101 is a monolithic apparatus. The sleeve may be formed from a single material. The sleeve may be formed as separate elements and thereafter integrally assembled. The sleeve may be formed of two or more materials to achieve different performance characteristics. For example, it may be desirable to use a more rigid material in regions where the sleeve is intended to encounter greater forces and more flexible material around regions corresponding to complex bends in the anatomy. This may allow for more uniform distribution of compressive forces and reducing kinks. Alternatively, it may be desirable to provide a more rigid material in the sleeve in a region adjacent a fluid bladder and corresponding to a tight radius on the body. For example, a fluid bladder would likely suffer from kinking (blockage of the fluid channel) if it were wrapped tightly in the armpit area. The likelihood of kinking increases when compressed the fluid bladder is subjected to large compression forces of the sleeve against the body. The likelihood of kinking increases even more in the case of a fluid bladder under the weight of the body, such as a fluid bladder on the back between an unconscious body and the ground. Techniques for reinforcing the heat transfer device and sleeve to reduce the likelihood of kinking are described in greater detail in U.S. application Ser. No. 12/939,986, the entire contents of which are incorporated herein for all purposes. Suitable materials for sleeve 101 include, but are not limited to, spun bonded material, hook and loop material, spun materials, woven and non-woven materials, laminates, and more. Suitable materials for sleeve 101 also include, but are not limited to, polymers such as nylon and poly(ethylene), and elastomers.

In various embodiments, the sleeve comprises a permanent antimicrobial finish to prevent mold growth, such as finishes made according to military specification MIL.STD.810D. The finish can be applied by placing the fabric in a chemical dip as is known in the art. In various embodiments, therapy wrap 17 includes a blood barrier to prevent contamination of heat transfer device 22 and/or sleeve 101. The barrier may also reduce the transmission of bacteria from patient to patient. For example, the inner surfaces of the pouches for receiving the heat transfer device may be blood-resistant. In another example, all or a portion of the sleeve may be covered by a blood-resistant material or coating. Examples of blood-resistant materials include nylon with a durable water repellency (DWR) coating, typically a ½ ounce polyurethane coating.

Exemplary sleeve 101 is configured to receive a flexible fluid bladder 32. The exemplary sleeve is also configured to receive a compressive bladder 30 to apply a compressive force to the body. The sleeve may include a pouch 102 for receiving one or more heat transfer devices 22, in particular a fluid bladder, such as disclosed by U.S. Pat. No. 7,896,910, the entire contents of which are incorporated herein for all purposes. One will appreciate from the description herein that other techniques can be used to attach the heat transfer device to the sleeve.

Pouches 102 may be selectively positioned in predetermined locations on the therapy wrap. In other words, the pouches may be fixed into a position on the wrap based on parameters defined before use of the wrap. Such parameters may include user preferences or application demands. In various embodiments, the sleeve is configured to position a bladder in one of a plurality of predefined locations. The predefined locations may be determined by user preferences. For example, the sleeve may be configured to allow a user to adjust the bladder attachment locations based on his or her preferences. This may be accomplished, for example, by providing an adhesive or other mechanism to allow a user to define the possible location of pouches 102. In various embodiments, the predefined locations correspond to traditional thermal treatment locations on the body such as a joint, a muscle, or the chest. In various embodiments, the predefined locations correspond to key areas for core cooling of the body.

Exemplary pouch 102 is configured to allow heat transfer device 22 to float therein. In other words, beyond being confined in the pouch, there are no fixed connections between the members. This can provide a more evenly distributed compression around the heat transfer device, resulting in improved therapy of the body being treated. Further, in the case of a fluid bladder, there is less chance that a portion of the fluid flowpath will be blocked when the apparatus is improperly applied because the fluid bladder can move and shift within the pouch to relieve pressure.

Sleeve 101 may have a variety of shapes and sizes for applying to different anatomies. The sleeve may be shaped and configured for application to a mammal, and in various embodiments, a human. The therapy wrap(s) may be shaped for applying to and covering different parts of the body as would be appreciated by one of skill from the description herein. The parts of the body to receive treatment with the wrap include, but are not limited to, all or part of a torso, a thoracic region, a cranial region, a throat region, a limb (e.g. a thigh or arm), a heart region, a lung region, a chest region, a wrist, a foot, and a combination of the same.

The sleeve may be configured for positioning the heat transfer device adjacent selected portions of the patient's vascular system, for example, the heart, the femoral artery, the carotid artery, or the superior vena cava. In various respects, the therapy wrap may include components configured for applying to a small body parts such as a wrist. Aspects of the therapy wrap may be similar to the wrap disclosed by U.S. Patent Pub. No. 2001/0034546 A1 to Elkins, the entire contents of which is incorporated herein for all purposes by reference.

In various embodiments, the therapy wrap is dimensioned and configured to apply thermal therapy to portions of the body to enable core body cooling. In various embodiments, the therapy wrap is dimensioned and configured to apply cooling to portions of the body at risk of ischemia from ventricular fibrillation and the like.

Exemplary sleeve 101 is shaped and sized to cover the torso of a human. The sleeve includes a main body portion 105 for enclosing the torso of the human. Sleeve 101 further includes an upper portion 107 for covering the head 108 and/or neck 110 of the human. The sleeve may be configured to slip over the head and arms using techniques that would be understood from the description herein. For example, the head and arm holes may allow for stretching. Alternatively, the sleeve may be configured to wrap around the head, arms, and other appendages using straps similar to straps 120 described above.

In various embodiments, therapy wrap 17 is a unitary member. In various embodiments, sleeve 101 is a unitary member. In various embodiments, the sleeve comprises two or more independent parts. For example, upper portion 107 may be separate and removable from main body portion 105.

The main body portion includes arm holes 103 for allowing the body portion to fit snugly around the torso. Upper portion 107 includes a neck hole 112 for receiving a neck of the patient. In the exemplary embodiment, the upper portion and main body portion are configured to receive one or more heat transfer devices 22 for cooling the body. The upper portion also includes a flap 114 for attaching the neck portion to the main body portion. Various aspects of the sleeve shape and design are similar to the apparel disclosed by U.S. Pat. No. 7,107,629 to Miros et al., the entire contents of which are incorporated herein for all purposes by reference.

In the exemplary embodiment, flap 114 is configured to cool the body. The exemplary flap extends only along a front of the neck region. This flap shape reduces the amount of material necessary to attach the upper and body portions. The exemplary flap shape also corresponds to the regions of the neck where the veins and arteries connecting the body to the brain are most exposed, in particular the jugular vein. The exemplary flap also covers the thyroid region in the neck.

In various embodiments, the therapy wrap is adapted to provide a compressive force to all or a portion of the body. In various embodiments, the therapy wrap is adapted to provide a compressive force to all or a portion of a treatment area, defined as a site through which treatment is delivered. Wrap 17 may fasten tightly to the body and apply a compressive force using sleeve 101. In one embodiment, the heat transfer device includes a compressive bladder 30 for applying a compressive force. The compressive bladder may be positioned adjacent a fluid bladder or other cooling source, or the compressive bladder may be positioned remotely. In one embodiment, the wrap includes a compressive device for applying a compressive force directly to the body. The wrap may include a mechanical device for applying the compressive force. For example, the wrap may include a gas pressure bladder that “balloons” or distends against the body with addition of gas, an actuator assembly, a piston assembly, and the like.

In various embodiments, exemplary wrap 17 is customized for a particular body location. In this embodiment, different regions of the wrap receive different amounts of heating, cooling, and/or compression. The exemplary wrap is configured to apply little or no compressive force around the neck to avoid the risk of suffocation. By contrast, the exemplary wrap is configured to apply a compressive force to chest. In various embodiments, the wrap applies a compressive force to the chest between about 0.25 psig and about 5 psig during use. The exemplary wrap is configured to apply a relatively high compressive force to the shoulders. Exemplary wrap 17 is configured to provide customized cooling. A region corresponding to the lungs is configured to provide greater cooling than around the shoulders or sides of the body, for example.

Therapy wrap 17 is configured to deliver supplemental therapy to the body in addition to thermal therapy. To that end, the therapy wrap is configured to apply working elements of supplementary therapy device 13 to the patient.

Exemplary wrap 17 is configured to mount electroconductive members to body 15. The exemplary electroconductive members are flat electrodes 65. Exemplary sleeve 101 includes cut outs for receiving the electrodes. In various embodiments, the electrodes are integrated into sleeve 101. As shown in FIG. 10, the electrodes are positioned along an inner region of the sleeve, between where the heat transfer device 22 and body will be located. The exemplary electrode placement corresponds to a location of the heart when the wrap is applied to the body.

The wrap may include other features to aid in delivery of the supplemental therapy such as would be understood by one of skill in the art. For example, the wrap may include a channel for receiving wires connecting the electrodes and a wiring harness 112. The wrap may also include features to decrease the risk of physical and/or functional interference between the therapy devices. The exemplary wrap includes barriers (i.e. electrical insulators) for reducing the risk of delivering electrical stimulation to areas other than around the heart. As shown in FIG. 10, for example, a thin layer of material 109 separates heat transfer device 22 from electrodes 65. The material 109 may be electrically insulating, such to prevent any electrical charge from leaking to the fluid bladder. The material may be rigid to promote positive apposition of the electrodes to the body when placed in compression.

In various embodiments, sleeve 101 includes an attachment or electrode region 265 for electrodes 65 distinct from a thermal treatment region. The electrode region may include holes or cut outs for the electrodes. The electrode region may include attachment mechanisms for attaching or mounting the electrodes, such as a mechanical fastener, an adhesive, or other mechanical, chemical, electrical, or ionic means. In various embodiments, the fluid and compressive bladders do not overlap the electrode region. In various embodiments, the electrode region is electrically and physically isolated from the thermal therapy working elements and/or remainder of the wrap. The wrap may include electrical insulation to isolate the electrical activity of the electrodes thereby preventing significant electrical leakage to the remainder wrap or outside the target treatment area of the electrical stimulation device.

In various embodiments, sleeve 101 a second region associated with second therapy device 13 is physically, electrically, and/or chemically isolated from a first region associated with first therapy device 12. The two regions may be isolated using techniques understood by one of skill from the description herein. Physical isolation of the second region may include, but is not limited to, fluid separation, gas separation, thermal separation, and/or physical distance. In various embodiments, the first and second regions are thermally isolated such that no significant heat transfer occurs between the two. In an exemplary embodiment, the second therapy device working elements are positioned in therapy wrap 17 sufficiently remote from heat transfer device 22 such that heat transfer does not occur between the two during use. Thermal insulating materials may be used in place of or in combination with physical distance to achieve isolation. Similarly, an electrically isolated region may be achieved by physical separation (the electromagnetic interference being inversely proportional to distance) and/or use of electrically insulating materials.

A therapy wrap in accordance with various aspects of the invention includes a variable insulating layer. “Variable insulating” generally refers to providing an insulating element in targeted locations and/or providing a variable thermal resistivity along the wrap. The insulating layer may improve the performance of the wrap by, among other things, compensating for the temperature delta through heat transfer device 22 and/or protecting the body part in selected areas. The insulating layer may also serve to selectively reduce the temperature delta by insulating a portion of the flowpath from heat loss.

Suitable materials for the insulating member include, but are not limited to, a foam, a plastic, a fibrous material, and other insulating materials known in the art. For example, the insulating member may be composed of a fabric, spray-on rubber (e.g., poly(urethane)), glass fibers, and more. The insulating member may also include structures and configurations for controlled insulating effect. For example, in place of an insulating member of a solid material, a housing may be provided that encloses a defined volume of gas (e.g. air) of a known thermal resistance. In another example, the insulating member may comprise a bladder filled with a thermo-resistive gel with a predetermined thermal resistance value. The insulating member may be selected based on the material properties including, but not limited to, thermal resistance (R-value). Generally, the material properties, dimensions, and configuration are adjusted to provide the desired insulating of the wrap at the desired location and/or a variable amount of insulating. The insulating members and other features described above may be distinct from therapy wrap 17 or integrally formed with the wrap. The members and features may be integrally formed with the heat transfer device or other working elements.

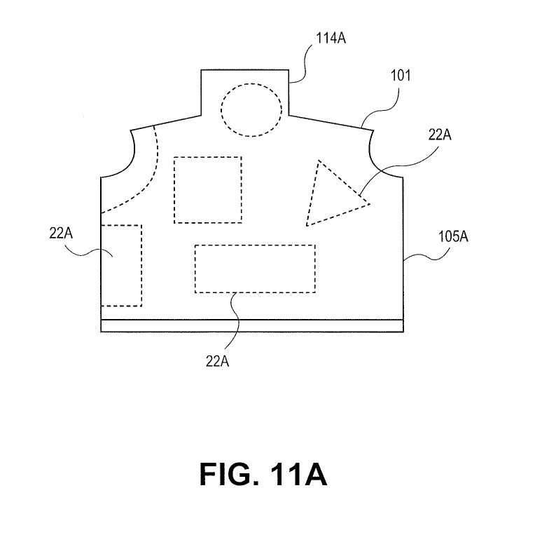

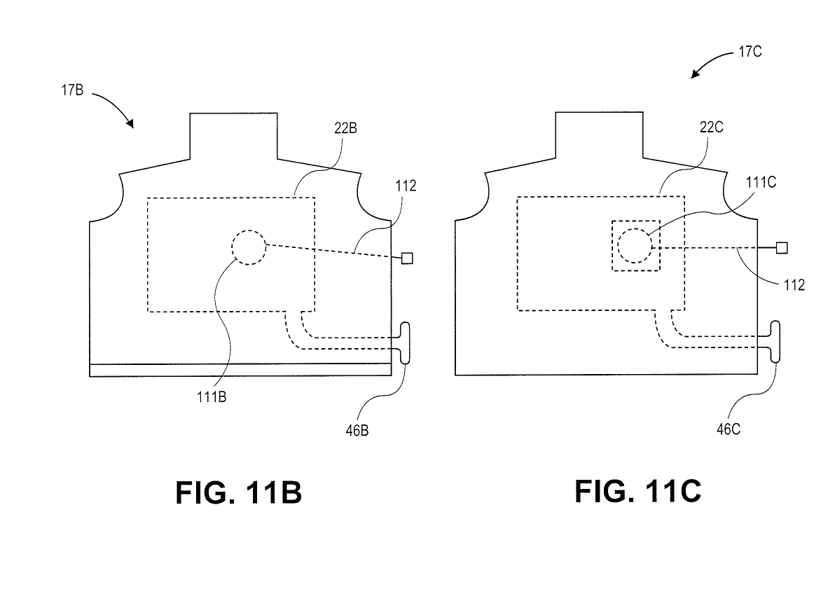

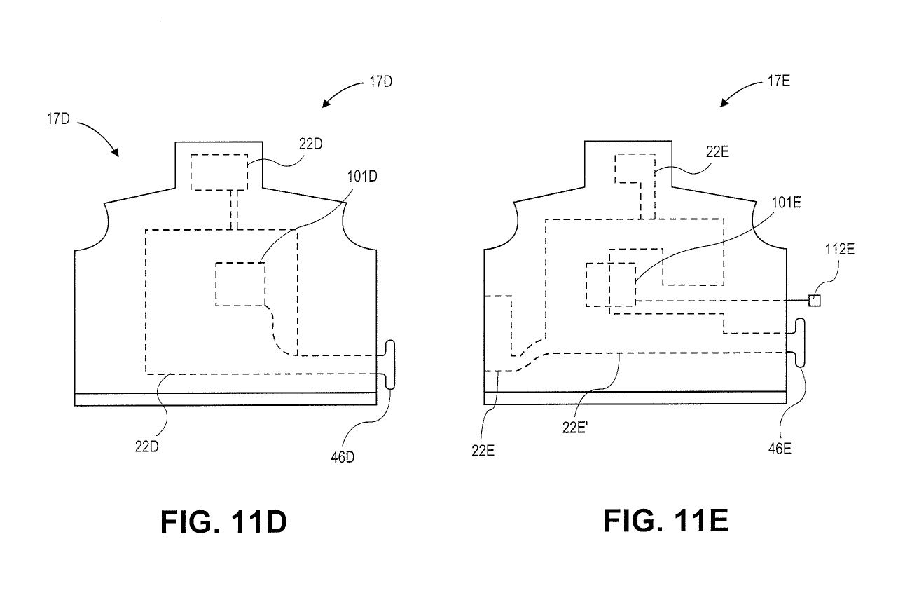

One of skill will appreciate from the description herein that the therapy wrap may be modified in other ways to promote the administration of different therapies. FIGS. 11A-11E illustrate a few, but not all, of the many possible configurations of system 10, and in particular therapy wrap 17. Like reference numerals have been used to describe like components. In operation and use, the different therapy wrap configurations are used in substantially the same manner as therapy wrap 17 discussed above.

FIG. 11A illustrates a therapy wrap 17a similar to therapy wrap 17. Therapy wrap 17a includes a plurality of heat transfer devices 22a. Therapy wrap 17a also includes a fastener 120 at a bottom portion for securing the wrap to the body. Any suitable fastener may be used such as an elastic band, belt, hook-and-loop fastener, or tie cord. The heat transfer devices are positioned in different locations around the body to which the therapy wrap is intended to be applied. The different shapes and sizes of the heat transfer devices allow for selective coverage of the body. FIG. 11A illustrates that the heat transfer devices can have a shape to conform to a part of the body. This preferential shape of the heat transfer devices may promote application of therapy wrap 17a around complex contours of the body. The shape heat transfer device may also have a shape that corresponds to a body part, for example, an area of an organ or joint.

FIG. 11B illustrates a therapy wrap 17b similar to therapy wrap 17. Therapy wrap 17b includes a single large heat transfer device 22b and a working element 111b of supplementary therapy device 13b. The working element may include, but is not limited to, an electrode, an actuator, a sensor, and the like. Working element 111b is positioned between a sleeve 101b, and heat transfer device 22b, and the body. The heat transfer device includes a gas pressure bladder for applying a compressive force against the body. In this configuration, the sleeve and compressive force aid in promoting contact between the working element and the body. Many applications can benefit from the improved contact between the working element and the body enabled by therapy wrap 17b, for example, achieving good electrical contact with electrodes. The sleeve and compressive bladder may also act to counter forces by the working element mechanically acting on the body.

In exemplary therapy wrap 17b, working element 111b and heat transfer device 22b are in overlapping relationship. The two elements, however, have separate connections to the external components such as the control units and power sources. The heat transfer device includes tubing and a multi-port manifold connector 46b similar to those described in U.S. Pat. No. 6,871,878 to Miros, the entire contents of which are incorporated herein for all purposes by reference. The connector exits the therapy wrap through an opening along a side of a bottom portion of the wrap. The exemplary therapy wrap includes an opening to allow the wiring for the working element to exit above the connector. In the exemplary embodiment, the working element is electronically operated and the wiring is a wiring harness 112. The above configuration allows a user to easily connect the heat transfer device and working element while positioning the body flat on its front or side. This can be advantageous, for example, when the body is unconscious.

FIG. 11C illustrates a therapy wrap 17c similar to therapy wrap 17 and 17b. Therapy wrap 17c includes a single heat transfer device covering a significant portion of a chest area of the body similar to therapy wrap 17b. Unlike therapy wrap 17b, however, therapy wrap 17c include a cut out or cavity for working element 111c. The exemplary cavity is configured to position the working element in a predetermined position along the body. The exemplary cavity includes optional fasteners for attaching the working element in the cavity.

FIG. 11D illustrates a therapy wrap 17d similar to therapy wrap 17. Therapy wrap 17d includes two heat transfer devices 22d. A first heat transfer devices 22d is positioned over a neck region of the body, and a second heat transfer devices 22d′ is positioned over a chest region of the body. The heat transfer devices have different sizes and shapes based on the parts of the body to which they are to be applied. Relative to the second heat transfer device, first heat transfer devices 22d has a smaller size and narrower shape corresponding to the neck.

The first and second heat transfer devices also are configured for different amounts of cooling therapy. In the exemplary wrap, the second heat transfer device over the chest region has greater cooling per square inch than first heat transfer device. This can be achieved as would be understood by one of skill from the description above.

The first and second heat transfer devices also are configured for different amounts of compressive therapy. In the exemplary wrap, first heat transfer device 22d positioned over the neck region does not include any compressive device to avoid choking the patient. By contrast, second heat transfer device 22d′ positioned over the chest is configured to apply a compressive force to the body in a range between about 0.25 psig and about 2 psig, preferably between about 0.25 psig and about 0.5 psig. In various embodiments, no compressive force is applied. This may be required if the patient is unconscious or is having trouble breathing.

In contrast to therapy wrap 17, therapy wrap 17d includes a working element 111d and heat transfer devices wired together. First heat transfer device 22d is serially connected to second heat transfer device 22c1′. The exemplary working element 111d of supplementary therapy device 13d is joined together with one of the heat transfer devices as a single assembly. The working element and heat transfer device may be assembled together using conventional techniques such as an adhesive or fastener. The wiring for working element 111d is bundled together with and routed through an opening in therapy wrap 17c1 with the heat transfer device tubing and wiring. One will appreciate that the working element and heat transfer devices may also be wired together.

FIG. 11E illustrates a therapy wrap 17e similar to therapy wrap 17. Therapy wrap 17e includes a plurality of heat transfer devices 22e and a supplementary therapy device working element 111e. The heat transfer devices have different shapes, sizes, and configurations. The heat transfer devices may be connected in series or in parallel. The heat transfer device 22e′ positioned over a central area of the chest includes a cut out region 115 where no cooling therapy is delivered to the body. Some or all of working element 111e can overlap the cut out region. In the exemplary embodiment, a portion of the working element overlaps or lies within the cut out region. This may be beneficial where it is desirable to adjust the interaction between the working element, heat transfer device, and/or body.

FIGS. 12A-12D are illustrative cross-sectional views of different configurations of the therapy wrap, looking from top to bottom, in accordance with the invention. The heat transfer devices are shown within a portion of a therapy wrap. Like reference numerals have been used to describe like components.

FIG. 12A illustrates therapy wrap 17 comprising a heat transfer device 22f including a fluid bladder 32f and gas pressure bladder 30f positioned inside a pouch of the therapy wrap. The exemplary therapy wrap is in the form of a sleeve similar to therapy wrap 17. The fluid bladder and gas pressure bladder include a matrix of connections 120. The exemplary connections are weld lines or points formed by RF welding.

The therapy wrap further includes a working element 111f of a second therapy device 13f. The exemplary working element overlaps the fluid bladder and gas pressure bladder in a lateral (lengthwise) direction. In a longitudinal (widthwise) direction, the working element is spaced apart from the fluid bladder. As used herein, lateral direction refers to a direction from left to right on the page or from one side of the body to the other when the wrap is applied to the body. Longitudinal direction refers to a direction from the body outward when the wrap is applied to the body. The exemplary working element is positioned in a wall of the therapy wrap.

FIG. 12B illustrates therapy wrap 17g comprising a heat transfer device 22g including a fluid bladder 32g and a gas pressure bladder 30g, and a working element 111g of a second therapy device. The exemplary working element is positioned directly adjacent the heat transfer device 22g, and in particular, between the heat transfer device and an inner surface of sleeve 101g.

Fluid bladder 32g extends the entire width of the heat transfer device along a lower portion but does not extend across the entire width in a portion of working element 111g. Instead, the fluid bladder includes a cut out region corresponding to the working element such that the heat transfer of the bladder does not interfere with the working element. Gas pressure bladder 30g extends only along a portion corresponding to the working element. Accordingly, the gas pressure bladder is configured to apply a compressive force to the body through the working element.

FIG. 12C illustrates therapy wrap 17h comprising a heat transfer device 22h including a fluid bladder 32h and a gas pressure bladder 30h, and a working element 111h of a second therapy device. Various aspects of therapy wrap 17h are similar to therapy wrap 17. Unlike therapy wrap 17, the working element shown in FIG. 12C is positioned outside a pouch of therapy wrap 17h. Specifically, the working element is positioned along an inner surface of sleeve 101h. In use, the exemplary working element is positioned on the body and then held in place by the force of the sleeve. Positioning of the working element may be aided by permanently or temporarily attaching the working element to the sleeve.

FIG. 12D illustrates therapy wrap 17i comprising a heat transfer device 22i including a fluid bladder 32i and a gas pressure bladder 30i, and a working element 111i of a second therapy device. The heat transfer device extends the entire width of the pouch 102i. The working element 111i is positioned within a second pouch 102i along an inner wall 112 of sleeve 101i.

FIG. 12E illustrates therapy wrap 17j comprising a heat transfer device 22j including a fluid bladder 32j and a gas pressure bladder 30j. The therapy wrap further includes a plurality of working elements 111j associated with a second therapy device. The exemplary working elements are electrodes configured for low level electrical stimulation such as for relieving muscle pain. The fluid bladder extends only along a portion of the width of the heat transfer device and pouch 102i. The exemplary gas pressure bladder is an expandable bladder configured to expand upon injection with a pressurized fluid. Suitable pressurized fluid includes, but is not limited to, pressurized air and helium. The expandable bladder may be a compliant bladder. In other words, the bladder may be configured to expand to a predetermined, controlled shape. As shown in FIG. 12E, the gas pressure bladder expands from a first position P1 to a second position P2. Expansion of the bladder promotes application of pressure on the body and/or working elements.

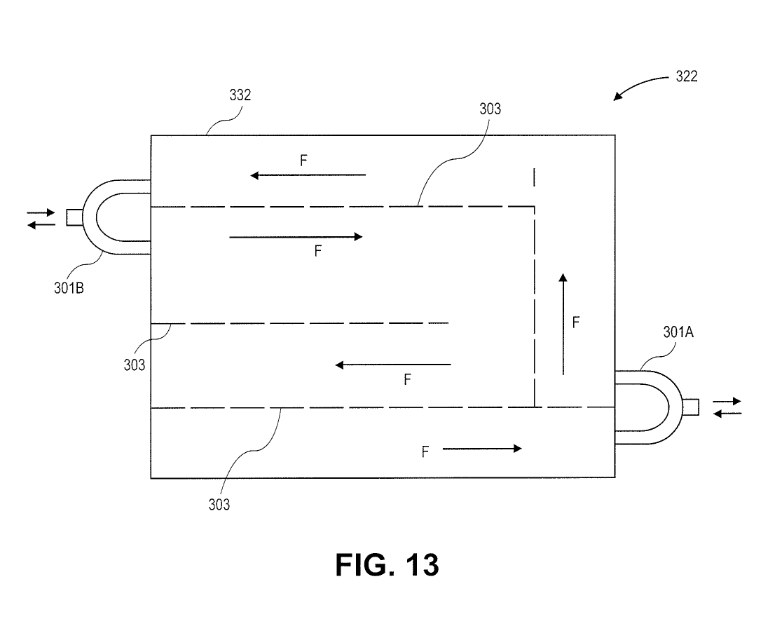

FIG. 13 illustrates a representative fluid flowpath in an exemplary fluid bladder 332. The fluid bladder includes a series of fences 303 and a border 305. Further details regarding fluid bladders with internal fluid flowpaths and their operation and manufacture are described in U.S. Pat. No. 7,198,093 to Elkins, the entire contents of which are incorporated herein for all purposes by reference.

The internal fences 303 define a flowpath F through bladder. The exemplary flowpath is somewhat circuitous. The heat transfer fluid thus flows through a relatively large area of heat transfer device 322. The exemplary fluid bladder includes one or more jumpers 301. Exemplary jumpers 301 are positioned along edges of the fluid bladder adjacent “dead ends” in the fluid flowpath. The jumpers include an inner lumen for channeling fluid flow and may be formed of a pipe, tube, or other fluidics. The jumpers also include a fluid port for introducing or removing fluid. In operation, the jumpers allow for the selective adjustment of the fluid flowpath. In a typical embodiment, the fluid flows into a first jumper 301a, turns a corner, and then flows into a second jumper 301b. The second jumper redirects the fluid back into the fluid chamber where it flows through the remainder of the fluid bladder. The warmed fluid then exits through first jumper 301a. In an alternative embodiment, fluid is introduced into the wrap through second jumper 301b and circulates through the fluid bladder with the aid of first jumper 301a. The jumpers may also be used to introduce another source of fluid midstream. In the first embodiment, for example, cooler feed fluid can be mixed with the circulated fluid or replace the circulated fluid before it is reintroduced into the fluid bladder. In this way, the jumper can be used to boost cooling performance.

In various embodiments, the location of the jumpers and/or manifold connector when the wrap is in position may be chosen to reduce interference with the body or other system components. The heat transfer device (heat exchanger) and jumpers may be oriented and positioned to enable easier connection of the device to the control unit. For example, it may be desirable to have the fluid ports oriented towards the side of the patient when the wrap is applied to the body. This makes the ports more accessible and avoids the risk of the fluid lines being pinched under the body's weight. In the case of an unconscious patient lying on the floor, the provision of extra fluid ports and/or placement in desirable locations avoids having to move the patient to connect the wrap to the control unit. The extra connection ports provided by the jumpers may also be beneficial in emergency and medical environments where the body cannot be easily moved. In an exemplary embodiment, the device includes jumpers configured to allow fluid connection to device from a top and bottom of the therapy device when it is positioned on the body. This configuration, for example, allows the body to be loaded into an ambulance head first or feet first without a long hose extension. The jumpers and/or manifold connector may extend from the side of the body, from the shoulder, downward from the waist, and other positions as would be understood by one of skill.

One will appreciate that the jumpers may be opened and closed to achieve many different flowpaths with a single configuration. One will also appreciate that the number and type of jumpers may be modified to adjust the flexibility of the system.

One will appreciate that any of the features of system 10 described herein may be manufactured using known techniques. Additionally, manufacturing techniques common in the polymer and semiconductor fields may be used such as etching, deposition, and lithography. Further details regarding the components and manufacturing techniques that may be used are disclosed in U.S. Pat. No. 7,198,093 to Elkins, the entire contents of which are incorporated herein for all purposes.

Although described in terms of a device for delivering electrical energy to the body, one will appreciate from the description herein that the same principles may be used to configure therapy wrap 17 for use with other therapeutic devices.

### V) Method of Administering Treatment

In various respects, system 10 is operated in a similar manner to operation of the respective therapy devices individually. In contrast to conventional devices, the shape and configuration of therapy wrap 17 allows the user to apply the components to the patient easier and faster. For example, in order to administer CPR, defibrillation, and cooling therapy to a patient with conventional devices, a user needs to perform a series of complicated steps including addition and removal of components. The steps would also be subject to greater risk of user error. Additionally, each device generally can only be applied to the body at a single time thus causing delays in administration of several therapies.

With particular reference to FIGS. 1, 8, 9, 10, and 14, system 10 allows for easier and more effective administration of thermal therapy with one or more other therapies. Various aspects of the invention relate to application of electric stimulation (e.g. a pulse) in combination with cooling therapy and optional compression. In various embodiments, the method relates to emergency medical treatment for a patient. For example, the patient may be suffering from ventricular fibrillation or stroke. In various embodiments, the method relates to deep core cooling of a patient during surgery and/or post-operative care (post-op).

Although the method of the invention will be described in terms of electrical stimulation in combination with cooling therapy, one will appreciate that the methods and systems of the invention may be configured for administering a variety of treatments to a patient. Suitable treatment settings include, but are not limited to, a clinic such as a rehabilitation or physical therapy clinic, an operating room (OR), a post-operative setting, a hospital, emergency medical care, and more. Various aspects of the invention may be particularly advantageous where portability is important.

Turning to FIGS. 8-10 and 14, an exemplary embodiment of the method using the system in accordance with the invention will now be described. The method will be described in connection with a system for administering cooling therapy and electric stimulation; however, the following description is not intended to be exhaustive or to limit the invention to the precise forms disclosed. To the contrary, one will appreciate that many modifications and variations are possible.

Prior to operation of system 10, the patient is prepared for the administration of treatment. The patient is generally prepared using conventional procedures relevant to the application. In the exemplary embodiment, the patient is suffering from ventricular fibrillation and the responding user intends to apply defibrillation and core body cooling as necessary. The user may have first attempted CPR. One will appreciate that the system may be equally effective for performing preparation techniques, such as CPR by integration or accommodation of a CPR device into the wrap. In an exemplary embodiment, the patient is prepared by removing clothing around the chest area and optionally applying a conductive gel to the skin over the heart region.

Once the patient is prepared, therapy wrap 17 is applied to the body. Exemplary therapy wrap 17 is designed and configured to be wrapped around the body like a vest. As shown in FIG. 8-10, main portion 105 of the therapy wrap can be positioned over the front of the patient and fastened in back with straps 120. Additional straps 120′ are pulled over the patient's shoulders and attached to the back of the wrap. The patient's head can be passed through a hole or slot in neck portion 107 of the wrap and arms are passed through holes or slots 103. Alternatively, the neck and arm portions can be configured with straps similar to main portion. The sleeve may be applied to the body by rolling it around the body from back-to-front like a blanket.

Next, therapy wrap 17 is connected to the remaining system components (shown in FIG. 9). The exemplary wrap includes an attachment section for attaching electrodes to an inner surface of sleeve 101. The exemplary system also includes sensors 44 for attaching to the sleeve. The wires are fed through an opening in a side of the sleeve and connected to the rest of the exemplary electric stimulation device. One or more heat transfer devices 22 are inserted into pouches in the sleeve as described above and connected to a control unit and cooling source through manifold connector 35. In various embodiments, the wrap is pre-assembled with one or more of the above components. For example, the therapy wrap may be pre-packaged with heat transfer device(s) and/or wiring.

In various embodiments, the system is configured to recognize the therapy wrap, heat transfer device, and/or supplementary therapy working element. For example, the components may include an identifier for identifying the wrap to the controller. Suitable identifiers include a bar code, RFID, or a unique electrical or mechanical configuration. For example, the therapy wrap may include a bar code that is scanned by the control unit. In another example, the respective element may include a code on the product label that is read by the user and entered into the system user interface.

One of skill will appreciate that, if the system recognizes what type of components are being used, the system can automatically tailor operation to the component. This information can be used in combination with the patient information. In various embodiments, a user selects a specific therapy wrap, heat transfer device, and working element from among a set of components. The system then recognizes which components have been selected and provides the associated, specific treatment. For example, a specific heat transfer device, therapy wrap, and working elements for treating a patient with cardiac arrest can be provided. When an EMS user encounters a patient suffering from cardiac arrest, the user selects these components and applies them to the patient. When the components are plugged into the system, the system recognizes that the patient is suffering cardiac arrest and proceeds to deliver treatment best tailored for that patient. In various embodiments, the system includes a plurality of treatment routines stored in a database.

At block 401, system 10 is primed and calibrated for thermal therapy. This operation may include a self-test to ensure that all the components are connected correctly. For example, the system can test for fluid or gas leaks. The system can also run a self-check of the electric stimulation device to confirm good contact with the body and to confirm the components are in working order. The system may also confirm that the electrodes are off.

Once the system is applied to the body and ready, the system is primed for operation. The priming includes charging the cooling source and power source for the electric stimulation device. The system also optionally runs some heat transfer fluid and gas through the heat transfer device to clear out the internal volumes.

In the meantime, the system begins monitoring the patient's vital signs by turning on the sensors at block 408. Suitable sensors include, but are not limited to, a temperature sensor, a pressure sensor, an EKG sensor, a pulse oximeter, and a blood pressure monitor. In the exemplary embodiment, the sensors are positioned between an inner surface of the therapy wrap and the body.

The system optionally includes sensors to monitor the system performance. For example, temperature sensors may be placed in the fluidics of the thermal therapy device and/or the cooling source. A pressure sensor may be used to monitor pressure in the compressive device.

The sensors acquire data at block 409 using conventional techniques. The acquired data may be displayed on an external monitor and/or logged in memory. In various embodiments, the data acquisition is performed based on various actions such as delivery of the electrical stimulation and at regular intervals thereafter. In various embodiments, the system logs information from the sensors and other components and stores the information in memory. The logged information may be used for feedback control, error logging, diagnostics, performance optimization, and the like. For example, the sensors and processing may be configured to recognize in operation of an electrode or blockage in the heat exchanger. The information may also be used for troubleshooting.

When the system is ready, for example when a sufficient charge is reached for the cooling source and/or electric stimulation power source, the system can be calibrated by the user using conventional techniques. The system may also be configured to self-calibrate.

At block 402, thermal therapy device 12 waits for a start point. Once the system reaches the start point, the thermal therapy process may begin. Waiting for the start point may comprise waiting for a desired control signal, referred to as S_{B,start}. The start control signal may be generated based on the patient's response, a manual input from a user, a system event, and the like. In an exemplary embodiment, the start control signal is generated when the cooling source reaches a desired temperature.

After calibration, the system also waits for a start point to begin the supplemental therapy at block 411. Similar to the thermal therapy process, the supplemental therapy may start based on receipt of a desired control signal, referred to as S_{A,start}. The start control signal may be related to the patient's response, a manual input from a user, a system event, and the like. The start control signal may be generated manually or automatically.

In an exemplary embodiment, the start control signal for the supplemental therapy device is generated when the power source (e.g. capacitor) is sufficiently charged or a predetermined time has elapsed since a patient event. In another example, the control signal is generated when the user pushes a button on the user interface 70. In another example, the user performs an action with the electrodes such as pulling a safety strip to close the circuit thereby triggering the control signal.

In various embodiments, a signal is transmitted to the supplementary therapy device controller 52 indicating that the body has reached a desired temperature, indicating a desired rhythm of delivery of chest compressions (CPR), indicating passage of a desired time interval, and the like. Upon receipt of the signal, the controller generates a start control signal to activate the supplementary therapy device 13. Examples of control signals that may be used to operate an electric stimulation device are disclosed by U.S. Patent Pub. No. 2008/0176199, the entire contents of which are incorporated herein for all purposes by reference.

Upon receipt of a desired control signal, S_{B,start}, thermal therapy device 12 is activated at blocks 403 and 404. Upon receipt of a desired control signal, S_{A,start}, supplementary therapy device 13 is activated at block 412.

In various embodiments, t_{A,start} and t_{B,start} are at the same. In various embodiments, t_{A,start} is prior to t_{B,start}. In various embodiments, t_{A,start} is after t_{B,start}. In various embodiments, t_{A,start} and t_{B,start} are contingent on each other. In various embodiments, t_{B,start} is generated a preset amount of time after t_{A,start} and/or a desired patient response (e.g. detection of a blood pulse). The system can also be configured so the user can input when the patient has recovered.

In the exemplary embodiment, activation of thermal therapy device 12 is contingent on completion of the electric stimulation therapy. Turning to block 412, exemplary system 10 switches to electrical stimulation mode after receiving the desired control signal. The exemplary electric stimulation device 50 is activated. Activation of the exemplary electrical stimulation mode includes stopping circulation of the heat exchange medium to the fluid bladder if it is on. Optionally, compression on the body part is increased such as by evacuating the fluid bladder and/or increasing the compressive force in the compressive bladder. As will be appreciated from the description herein, the thermal therapy device may be operated independently of the supplementary therapy device. The therapy devices may be operated simultaneously. At block 412, the system performs a final check to confirm good contact of the body with electrodes 65 (i.e. no short circuits and good apposition). Next, electrodes 65 are activated to apply an electrical stimulation pulse to the desired location on the body (e.g. the heart). The stimulation is applied in an otherwise conventional manner.

At block 413, supplementary therapy device 13 is turned off at t_{A,end}. The device may be deactivated based on receipt of a control stop signal, referred to as S_{A,stop}. In the exemplary case, the system confirms that the patient has recovered by detecting a desired signal. The signal may include, but is not limited to, the presence of a pulse, a desired ECG, or a manual input from the user. Examples of control signals that may be used to operate an electric stimulation device are disclosed by U.S. Patent Pub. No. 2010/0121392, the entire contents of which are incorporated herein for all purposes by reference.

In an exemplary embodiment, receipt of a desired control signal S_{A,stop} is the start signal S_{B,start} for thermal therapy device 12. After the start point (t_{A,start}) has been reached, thermal therapy device 12 turns on. In the exemplary method, the system begins applying thermal therapy after the patient has recovered using the supplementary therapy and the electrodes are turned off.

At block 403, control unit 27 delivers pressurized gas to heat transfer device 22, namely, the gas pressure bladder 30. The pressure in the gas pressure bladder exerts a compressive force on the body. In the exemplary system, once the gas pressure bladder is filled, the system generally maintains the pressure in the bladder by applying a backpressure.

At block 404, control unit 27 activates pump 25 to circulate cooled fluid to heat transfer device 22, namely, fluid bladder 32. Circulation of the cooled fluid causes heat transfer with the body. In various embodiments the thermal therapy device is operated under sufficient conditions to lower the body temperature below 95 degrees F., below 90 degrees F., below 80 degrees F., below 70 degrees F., below 60 degrees F., or below 50 degrees F. In various embodiments the thermal therapy device is operated under sufficient conditions to induce mild hypothermia. In various embodiments, the thermal therapy device is operated under sufficient conditions to lower the body temperature to about 50 degrees F. In various embodiments, the thermal therapy device is operated under sufficient conditions to lower the body temperature to between about 90 degrees F. and about 94 degrees F. In various embodiments, the thermal therapy device is operated under sufficient conditions to lower the body temperature in uniform increments. In various embodiments, the thermal therapy device is operated under sufficient conditions to lower the body temperature at a linear rate. In various embodiments, the body temperature is decreased by a few degrees each minute. In various embodiments, the thermal therapy device is operated under sufficient conditions to limit substantial increases in the core body temperature and/or mild hyperthermia. In various embodiments, the body temperature is decreased by at least 5 degrees F. per minute, preferably between about 5 and about 20 degrees F. per minute, more preferably between about 5 and about 10 degrees F. per minute. In various embodiments, the body temperature is decreased by between about 2 and about 10 degrees F. per hour, preferably about 5 degrees F. per hour. In various embodiments, the thermal therapy device is operated under sufficient conditions to lower the body temperature at a first rate for a first period of time and then a faster second rate thereafter. In various embodiments, the thermal therapy device is operated under sufficient conditions to lower the body temperature at a first rate for a first period of time and then a slower second rate thereafter.

In various embodiments, the thermal therapy device is cycled through different treatment conditions. For example, the device may be treated at one temperature for a first period of time and then treated at a lower temperature for a second period of time. In various embodiments, the body is cooled gradually, maintained at a predetermined temperature, and then restored to normal temperature gradually. In various embodiments, the thermal therapy device is configured to induce hypothermia. The device may apply different levels of cooling in different regions or to different body parts. For example, the device may apply greater cooling to the chest area than the wrist area to lower the body's thermoregulation defenses. The device may gradually even out the difference in temperatures as the body approaches the desired internal body temperature.

At block 405, the system waits for an end point t_{B,end}. In an exemplary embodiment, the system waits for a desired control stop signal, referred to as S_{B,stop}. Upon receipt of the desired signal, thermal therapy device 12 turns off. The desired signal may include, but is not limited to, information related to the system or patient response. The desired signal may be generated when core cooling of the patient has been achieved. Core cooling can be determined when the sensors indicate that the body temperature has reached a predetermined level. Core cooling can also be determined using known information in combination with the system operating conditions. For example, the user can input the patient's characteristics, and based on this information the system can determine that sufficient core cooling has been achieved using information related to the system performance. The patient characteristics may include height, weight, fitness level, condition or injury, and more. The information related to the system performance may include elapsed time, flow rate, temperature drop of the cooling source, and more. In various embodiments, the thermal therapy device runs until the user manually turns it off (e.g. using the control panel or disconnecting the wrap) or the cooling source has warmed above a threshold level where it cannot provide further cooling. In various embodiments, the system runs a shut down routine. The shut down routine may include a warm-up routine whereby the temperature in the therapy wrap is gradually restored to normal.

Next, the thermal therapy device turns off. At block 406, the control unit stops the flow of gas and/or application of backpressure to the gas pressure bladder. At block 407, the control unit stops the circulation of fluid to the fluid bladder. In various embodiments, the body temperature is gradually restored to its normal temperature.

After the thermal therapy and supplementary therapy is complete, sensors 44 are turned off at block 410. In various embodiments, the system delivers a signal to the therapy wrap and/or heat transfer device related to its use. The information may include an indication that the component has been used and under what conditions. This information can be stored in memory for later retrieval, for example, to pass to a doctor once a patient reaches the hospital, for optimizing, and/or troubleshooting.

One will appreciate that a number of the operations described above can be modified and performed in differing order. For example, the start and stop points for the thermal therapy and supplementary therapy may vary. In various embodiments, the therapy system is operated by applying thermal therapy and electrical stimulation intermittently. The thermal therapy and electrical stimulation may be applied sequentially or alternatively. The periods for application of thermal therapy and electrical stimulation may overlap each other. In various embodiments, the thermal therapy is applied before and after the electrical stimulation. The thermal therapy applied after the electric stimulation is performed under different conditions than before the electrical stimulation. In various embodiments, the system is configured to administer a plurality of supplementary therapies.

### VI) Other Systems and Features

One of skill will appreciate from the foregoing a number of other modifications and variations within the scope of the invention.

Various aspects of the invention are directed to a system for providing thermal therapy in combination with cardiac pacing. In various embodiments, electrodes 65 and the associated hardware are configured for cardiac pacing in an otherwise conventional manner. In various embodiments, the electrodes are configured for delivering an electrical stimulation pulse and cardiac pacing. In various embodiments, separate electrodes and circuitry are provided for pacing and electrical stimulation.

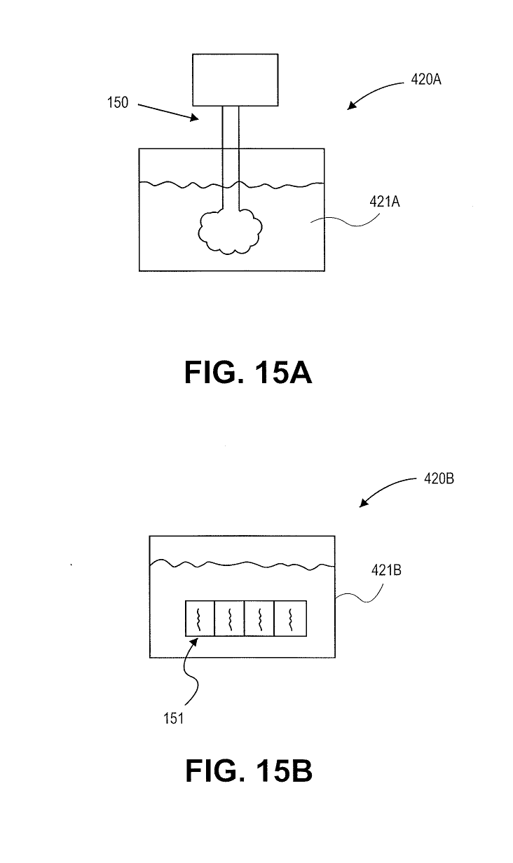

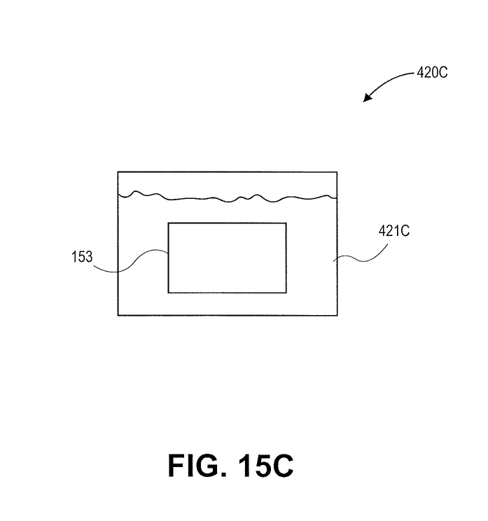

FIGS. 15A, 15B, and 15C illustrate variations of cooling source 20 described above. FIG. 15A illustrates a thermoelectric-based cooling source 420a. In various respects, cooling source 420a is similar to cooling source 20.

Cooling source 420a includes a reservoir 421a filled with a heat transfer fluid. Unlike cooling source 20, a thermoelectric cooler 150 extends into reservoir 421a for cooling. A distal end of the thermoelectric cooler positioned in the reservoir is maintained at a low temperature. The thermoelectric cooler may be cooled using otherwise known techniques. In various embodiments, the thermoelectric cooler is provided in combination with cooling source 20. Thermoelectric cooling source 420a is configured to provide near instantaneous cooling until primary cooling source 20 (e.g. an ice bath) is available. The control unit may be configured to monitor the temperature of the primary cooling means and transition from the thermoelectric cooler as necessary.

The exemplary thermoelectric system 420a generally benefits from faster cooling than cooling source 20. This can be critical for applications where cooling is needed on-demand, such as emergency medical care. Additionally, unlike an ice bath that needs to be refreshed periodically, cooling source 420a can be plugged in when needed and turned off when not needed. Since emergency medical response teams typically only respond to a few cases of cardiac arrest each month, it can be cumbersome to keep an ice-based cooling source ready. The electric activated cooling source overcomes this problem. The cooling source could also be a compressor-based cooling system or other electromechanical cooling system.

FIG. 15B illustrates another cooling system 420b having alternative cooling structures. In various respects, cooling source 420b is similar to cooling source 20. Cooling source 420b includes a reservoir 421b filled with a heat transfer fluid. Unlike cooling source 20, cooling source 420b is cooled by a chemical-based cooling element 151, which in various respects does not include a cooling fluid. An exemplar of a chemical cooler is a cold pack with chemical reactants separated by a barrier. A typical cold pack works through the reaction of gel or water with ammonium nitrate or ammonium chloride. The pack is activated by breaking or removing the barrier, which causes the chemicals to mix. When activated, the endothermic reaction draws all the heat from the water or gel. Once activated, the pack is dropped into the reservoir. The cooling source 420b may be provided alone or in combination with a conventional ice bath such as cooling source 20. Such chemical-based cooling may cool the heat transfer fluid reservoir quicker than ice baths. Similar to the electric-based cooling described above, the chemical-based cooling also has the advantage of allowing a user to activate the cooling source only when necessary.

In an exemplary embodiment, the therapy wrap is configured for passive control of cooling. The chemical cold pack is configured to maintain a temperature of about 45 degrees F., for example. The cold pack may be inserted directly into the sleeve or placed into the reservoir for flowing heat transfer fluid to a heat transfer device in the sleeve.

FIG. 15C illustrates another cooling system 420c having non-fluid cooling structures. In various respects, cooling source 420c is similar to cooling source 20. Cooling source 420c includes a reservoir 421c filled with a heat transfer fluid. Unlike cooling source 20 which is generally cooled with ice, cooling source 420c is cooled with self-contained, cooled elements 153. The cooled elements may include, for example, an herbal cold pack made of herbs and grains that retain cold, cooled gel encased in plastic, and the like.

Any of the above cooling source embodiments, may include other features as would be understood in the art from the description herein to aid in cooling. In various embodiments, the reservoir includes a mixer to mix the heat transfer fluid thereby avoiding cold spots and freezing. In various embodiments, the system is configured to mount the cooling source directly to therapy wrap 17. For example, the therapy wrap may include a pouch for receiving a chemical-based cold pack to eliminate the need for a reservoir and fluidics. In various embodiments, the chemical-based cold pack freezing temperature and/or heat transfer rate is selected to reduce the risk of tissue damage.

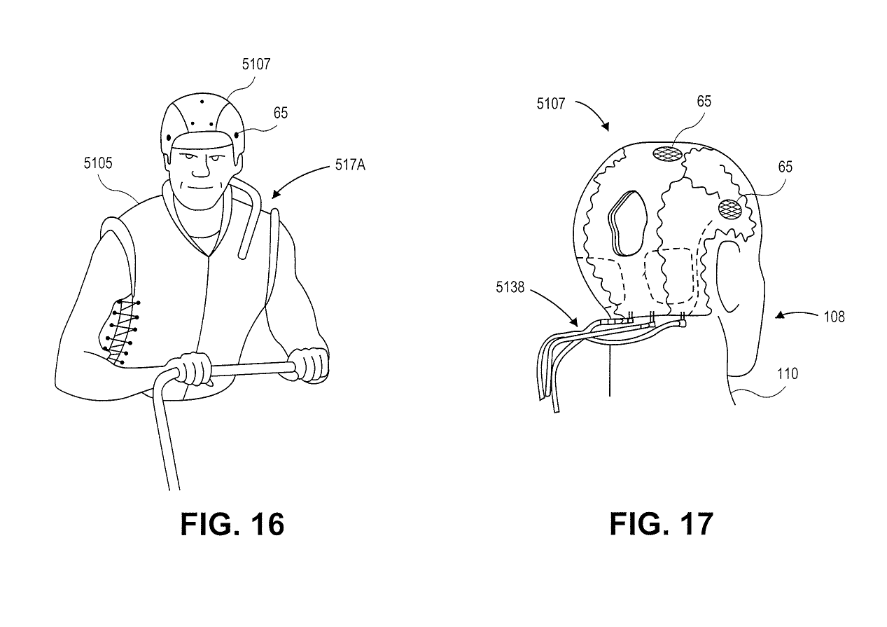

FIGS. 16-20 illustrate variations of therapy wrap 17 and/or sleeve 101 in accordance with the invention. Like reference numerals have been used to describe like components. In operation and use, the different therapy wrap configurations are used in substantially the same manner as therapy wrap 17 discussed above.

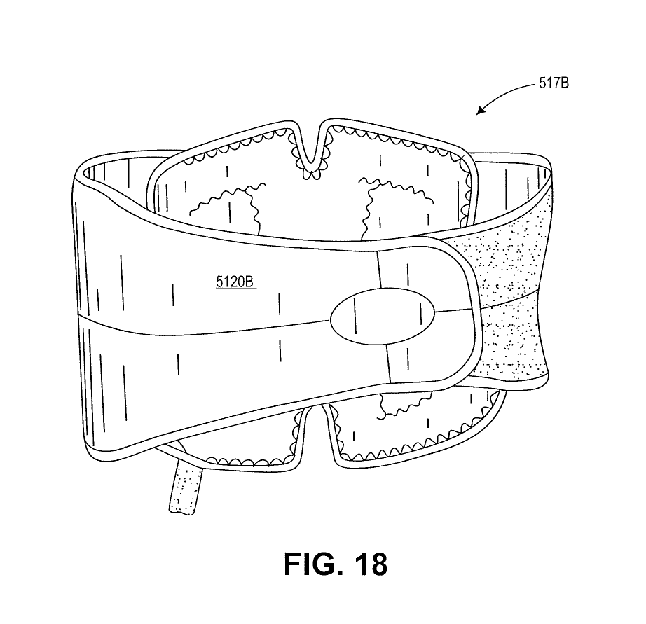

FIGS. 16 and 17 illustrates a therapy wrap 517a similar to therapy wrap 17. FIG. 16 illustrates therapy wrap 517a applied to a user performing exercise. The therapy wrap includes a vest component 5105 and a skull cap or helmet component 5107. In various respects, vest component 5105 and helmet component 5107 are configured and operated similar to sleeve body 105 and sleeve upper portion 5107, respectively. Unlike sleeve 101, vest component 5105 is shaped and configured for applying like vest or apparel. Helmet component 5107 is shaped and configured for close fitting to a head of mammal, in the exemplary case, a human. It has been found that cooling of the brain can significantly reduce the risk of long-term loss from cardiac arrest and ventricular fibrillation. Accordingly, the helmet component may be useful for cooling the brain of a person being treated for these events.

FIG. 17 is an enlarged view of the helmet component 5107 of FIG. 16. The helmet includes a plurality of connections in the back. The connections may include fluid and/or electrical connections. The exemplary helmet component includes a heat transfer device and an electric stimulation device. Unlike therapy wrap 17 described above, the two portions 5105 and 5107 of therapy wrap 517a are fluidly connected by an external tube and fluidics 5138. In various embodiments, the helmet may include a plurality of electrodes inside the helmet component configured for neural stimulation.

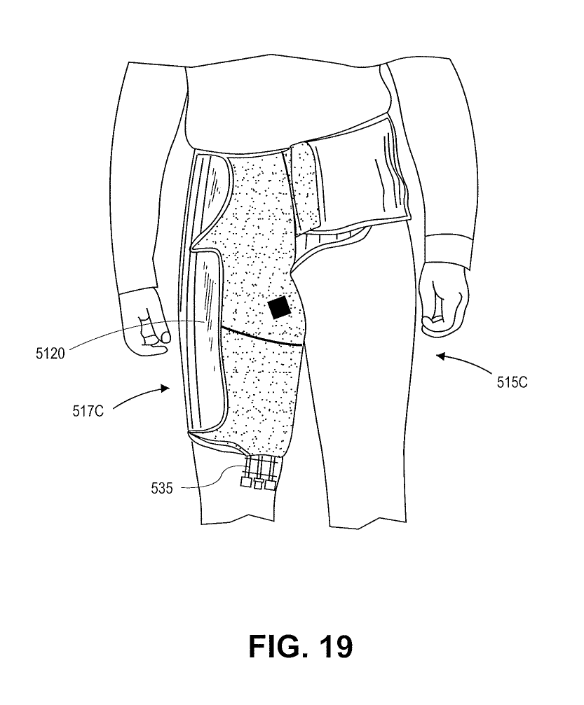

FIG. 18 illustrates another embodiment of the therapy wrap in accordance with the invention. In various respects, therapy wrap 517b similar to therapy wrap 17. Therapy wrap 517b is shown in a fastened configuration with the ends fastened together and ready for use. The wrap is configured for wrapping the waist or lower portion of a torso of a patient. The wrap further includes straps 5120b to allow for repositioning around the upper torso of the patient.

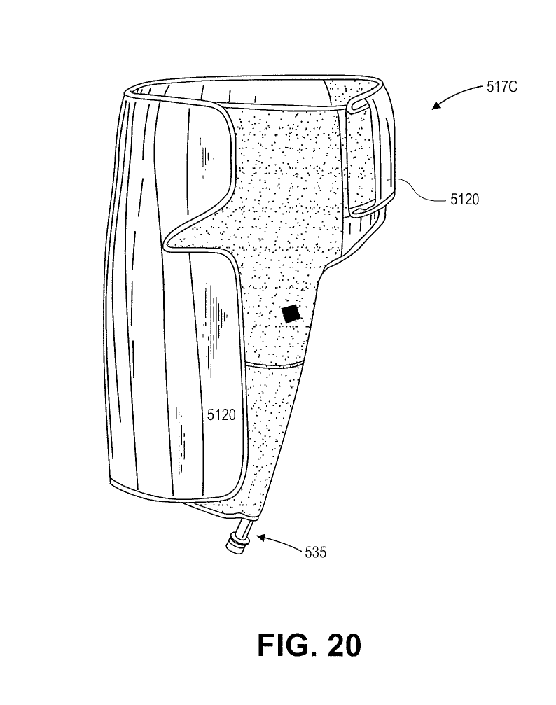

FIGS. 19-20 illustrate another embodiment of the therapy wrap applied to a body. In various respects, therapy wrap 517c similar to therapy wrap 17, except therapy wrap 517c is shaped and configured for applying to a lower portion of body 515c. In the exemplary case, the wrap is configured for wrapping around a hip, groin, and leg. The therapy wrap includes a manifold connector 535 and attachment straps 5120.

One of skill in the art will appreciate that a number of other features and modifications are within the scope of the invention. For example, the control system and sensors may be modified to perform different functions.

As described above, system 10 may include one or more sensors 44. Suitable sensors include, but are not limited to, pressure transducers and temperature sensors. The system may also include a timer and system clock.

In various embodiments, the system includes memory and software programming. The programming may be embedded in hardware such as in non-volatile memory. The system may include firmware.

The system may be preprogrammed to provide a variety of predetermined therapy procedures. For example, the system may be preprogrammed with set therapy routines. When administering treatment, one of the predetermined routines is automatically or manually selected for operation. In various embodiments, the system is programmed to deliver electrical stimulation based on the specific patient indications. Exemplars of methods for providing customized electrical stimulation are disclosed by U.S. Pub. Nos. 2010/0318145, 2010/031814, and 2010/0318144, the entire contents of which are incorporated for all purposes by reference.

In various embodiments, the system includes a treatment wizard. The system may be loaded with a treatment algorithm and selects a treatment routine based on a user's response to various questions. In various embodiments, the system includes programming for customized thermal therapy treatment.

In various embodiments, the system monitors the patient during treatment and then uses the gathered information to adjust the treatment protocols during treatment based on the patient's response. An exemplar of a method for adjusting electrical stimulation based on patient response is disclosed by U.S. Pub. No. US 2009/0270930, the entire contents of which is incorporated for all purposes by reference.

In various embodiments, the system is preprogrammed with compression treatment programs. For example, the system may be loaded with a program to deliver compression therapy in the form of alternating intervals of compression on and compression off for a specific injury.

As will be clear from the above example, the insulating layer may be a separately-formed, independent member for use with a variety of temperature-controlled therapy systems in accordance with the invention.

Variations and modifications of any of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

For convenience in explanation and accurate definition in the appended claims, the terms “up” or “upper”, “down” or “lower”, “inside” and “outside” are used to describe features of the present invention with reference to the positions of such features as displayed in the figures.

In many respects the modifications of the various figures resemble those of preceding modifications and the same reference numerals followed by apostrophes or subscripts “a”, “b”, “c”, and “d” designate corresponding parts.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A system for providing treatment to an animate body, the system comprising:
a first therapy wrap comprising a heat transfer device adapted to exchange heat with the animate body, the heat transfer device comprising an inner fluid bladder and an outer gas pressure bladder;
a second therapy wrap comprising a gas pressure bladder configured to apply a compressive force to the animate body, wherein the second therapy wrap does not provide a thermal therapy to the animate body; and
a cooling source comprising a reservoir configured to hold a heat transfer fluid, one or more pumps configured to pressurize both the outer gas pressure bladder of the first therapy wrap and the gas pressure bladder of the second therapy wrap and to circulate the heat transfer fluid between the inner fluid bladder of the first fluid bladder and the reservoir, and a control unit in communication with a user interface, the control unit programmed to control the circulation of the heat transfer fluid and the pressurization of the outer gas pressure bladder of the first therapy wrap and the gas pressure bladder of the second therapy wrap.

2. The system of claim 1, wherein the cooling source further comprises one or more self-contained cooling elements configured to be disposed in the reservoir.

3. The system of claim 2, wherein the one or more self-contained cooling elements are ice packs.

4. The system of claim 1, wherein the gas pressure bladder of the second therapy wrap is configured into a pressure cuff.

5. The system of claim 1, wherein the control unit is programmed to pressurize the outer gas pressure bladder of the first therapy wrap and the gas pressure bladder of the second therapy wrap to between 0.25 psig and 20 psig.

6. The system of claim 1, wherein the control unit is programmed to pressurize the outer gas pressure bladder of the first therapy wrap and the gas pressure bladder of the second therapy wrap to between 0.25 psig and 5 psig.

7. The system of claim 1, wherein the control unit is programmed to pressurize the outer gas pressure bladder of the first therapy wrap and the gas pressure bladder of the second therapy wrap to between 0.25 psig and 1.5 psig.

8. The system of claim 1, further comprising one or more sensors configured to acquire and send data to the control unit, wherein the control unit is further programmed to process the data from the one or more sensors.

9. The system of claim 8, wherein the one or more sensors are configured to acquire and send information from the system.

10. The system of claim 8, wherein the one or more sensors are configured to monitor the system performance.

11. The system of claim 8, wherein the one or more sensors are configured to acquire and send information from the animate body.

12. The system of claim 8, wherein the control unit is further programmed to adjust operation of the therapy wrap or control system based on the data from the one or more sensors.

13. The system of claim 8 wherein the control unit is programmed to use the sensor data to operate the system to produce a desired therapeutic result.

14. The system of claim 8, wherein the control unit is programmed to use the sensor data for diagnostic or troubleshooting functions.

15. The system of claim 8, wherein the one or more sensors includes a temperature sensor.

16. The system of claim 8, wherein the one or more sensors are configured to acquire and send one or more of the following: fluid inlet temperature, fluid outlet temperature, and fluid flow rate.

17. The system of claim 8, wherein the one or more sensors are located in or proximate to the reservoir.

18. The system of claim 8, wherein the one or more sensors includes a pressure sensor.

19. The system of claim 8, wherein the control unit is programmed to determine whether a leak or blockage is present based on the data from the one or more sensors.

20. The system of claim 8, wherein the control unit is programmed to determine whether fluid temperature is too high or too low based on the data from the one or more sensors.

21. The system of claim 1, further comprising a timer in communication with the control unit.

22. The system of claim 1, wherein the control unit is programmed with one or more compression treatment programs.

23. The system of claim 22, wherein one of the compression treatment programs includes alternating intervals of compression on and compression off.

24. The system of claim 1, wherein the first therapy wrap has a first portion configured to wrap around the patient's upper thigh and a second portion configured to wrap around the patient's hips.

25. The system of claim 1, wherein the cooling source is configured to be powered by an external electric power source.

26. The system of claim 1 wherein the second therapy wrap is configured to conform to the calf.

27. The system of claim 1 wherein the second therapy wrap is configured to conform to the foot.
